(19) United States
(12) Patent Application Publication
(10) Pub. No.: US 2025/0262251 A1
(43) Pub. Date: Aug. 21, 2025

(54) THERARONSTIC APPROACH FOR INFLAMMATORY BOWEL DISEASE-ASSOCIATED SPONDYLOARTHRITIS

(71) Applicant: Cornell University, Ithaca, NY (US)

(72) Inventors: Randy Longman, New Rochelle, NY (US); Svetlana Lima, Ithaca, NY (US)

(21) Appl. No.: 19/054,625

(22) Filed: Feb. 14, 2025

Related U.S. Application Data
(60) Provisional application No. 63/554,616, filed on Feb. 16, 2024.

(51) Int. Cl. A61K 35/74; A61P 1/00; C12Q 1/689

(52) U.S. Cl. CPC A61K 35/74; A61P 1/00; C12Q 1/689; C12Q 2600/112

(57) ABSTRACT

Provided are methods for treating an individual who has inflammatory bowel diseases (IBD and) spondyloarthritis by selecting the individual based on a determination that that the gastrointestinal system of the individual comprises a microbiome lacking bacteria that provide functional folate trap, and administering to the individual sulfasalazine and bacteria that include a functional folate trap to thereby treat the IBD.

### CROSS-REFERENCE TO RELATED APPLICATION

This application claims the benefit of priority to U.S. provisional patent application No. 63/554,616, filed Feb. 16, 2024, the entire disclosure of which is incorporated herein by reference.

### STATEMENT REGARDING FEDERALLY SPONSORED RESEARCH OR DEVELOPMENT

This invention was made with government support under grants R01 DK114252 and DK 120985 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Feb. 14, 2025, is named “018617.01851.xml”, and is 6,208 bytes in size.

### RELATED INFORMATION

Peripheral and axial spondyloarthritis (SpA) are the most commonly reported extra-intestinal manifestations of inflammatory bowel disease (IBD). Using diagnostic criteria established by the Assessment of SpondyloArthritis International Society (ASAS), population cohorts estimate an 8 and 12% prevalence of concomitant axial or peripheral SpA (pSpA), respectively, in patients with IBD^{1,2}. Limitations in the characterization of SpA disease activity in IBD cohorts has led to significant heterogeneity in the estimates of disease prevalence³⁻⁷. Furthermore, evidence supporting distinct genetic, cellular, and microbial factors which overlap between IBD and SpA highlights the potential for IBD-associated SpA as a separate clinicopathologic entity⁸; however, a better understanding of this biology is needed to guide more targeted and effective use of therapy.

Characteristic alterations in the intestinal microbiome associated with IBD are thought to be a contributor to SpA. Although clinical studies did not validate early associations of SpA with Klebsiella sero-reactivity, recent studies using advanced sequencing technology to provide a more complete microbiome analysis have identified potential microbial targets in subjects with IBD-SpA⁹⁻¹¹. Specifically, adherent-invasive Escherichia coli isolates were found to be expanded in Crohn's associated pSpA¹² and have been shown to promote T cell inflammation¹³. Gut dysbiosis in primarily HLA-B27 positive SpA patients was characterized by decreased microbial diversity, reduced Faecalibacterium prausnitzii, and increased levels of Ruminococcus gnavus that correlated with SpA disease activity in a subset of patients with a history of IBD^{11,14}.

Sufasalazine (SAS) is one of the earliest medications with demonstrated efficacy for induction and maintenance therapy in IBD¹⁵⁻¹⁸. SAS was designed as a prodrug that consists of sulfapyridine (SP) and 5-aminosalicylate (5-ASA) linked by a diazo bond, which prevents absorption in the proximal intestine¹⁹. Azoreductases produced by the colonic microbiota cleave the diazo bond and release SP and 5-ASA into the large intestine²⁰. While SP can be absorbed and may lead to systemic side effects, 5-ASA remains in the intestine and promotes mucosal healing^{21,22}; however, SAS, and not 5-ASA alone, is effective for the treatment of peripheral symptoms of SpA²³.

The anti-bacterial capability of SP to disrupt bacterial synthesis of folate has led to the hypothesis that the microbiome may be important for understanding the efficacy of SAS. Early studies in patients with rheumatoid arthritis²⁴ and mouse models²⁵ allude to the potential effects of SAS on the intestinal microbiome. Sulfamethoxazole, a drug with a similar mode of action to SP, was recently found to modulate E. coli metabolic capacity via folate stress, leading to the production of bacterial secondary metabolites with anti-inflammatory properties²⁶. However, the impact of the microbiome on the efficacy of SAS therapy for IBD-SpA has not yet been explored. There is accordingly an ongoing and unmet need for improved patient identification for treating IBD-SpA, and methods of treating the IBD-SpA based on analysis of IBD-SpA patient samples. The present disclosure is pertinent to this need.

### BRIEF SUMMARY

The present disclosure provides a prospective assessment of the clinical and microbiome characteristics associated with the efficacy of SAS therapy in subjects with active IBD-associated pSpA (IBD-pSpA). The Bath Ankylosing Spondylitis Disease Activity Index (BASDAI) is a patient-reported tool that has been clinically validated for assessment of inflammatory activity and responsiveness to therapy in axial SpA^{12, 27, 28} and pSpA²⁹. Another SpA disease activity index, the Ankylosing Spondylitis Disease Activity Score (ASDAS) includes patient-reported symptoms, a global activity score, and objective serum markers of inflammation such as C-reactive protein (CRP) or erythrocyte sedimentations rate (ESR)³⁰. Fecal microbiome analysis revealed a baseline gut bacterial signature associated with this clinical improvement in joint symptoms. Metabolic and transcriptional profiling identified the mechanistic regulation of microbial butyrate production by SAS in F. prausnitzii, a taxa associated with clinical response. The disclosure demonstrates a role for drug-microbiome interactions in regulating the clinical efficacy of SAS therapy for IBD-pSpA and provides a clinical model for further investigation of the biology of IBD-SpA as a distinct clinicopathologic entity. The disclosure also provides in examples a description of treatment of 22 IBD-pSpA subjects with sulfasalazine and identifies clinical responders with a gut microbiome enriched in Faecalibacterium prausnitzii and the capacity for butyrate production. The disclosure reveals that sulfapyridine promotes butyrate production and transcription of the butyrate synthesis gene but in F. prausnitzii in vitro, which is suppressed by excess folate. Sulfasalazine therapy enhances fecal butyrate production and limits colitis in wild-type and gnotobiotic mice colonized with responder, but not non-responder, microbiomes. The disclosure demonstrates that F. prausnitzii is sufficient to restore sulfasalazine protection from colitis in gnotobiotic mice colonized with non-responder microbiomes. The disclosure thus reveals a mechanistic link between the efficacy of sulfasalazine therapy and the gut microbiome which supports the presently described diagnostic and therapeutic approaches for treating IBD-pSpA.

### DESCRIPTION OF FIGURES

The patent or application file contains at least one drawing executed in color. Copies of this patent or patent application publication with color drawing(s) will be provided by the Office upon request and payment of the necessary fee.

FIG. 1. The gut microbiome stratifies sulfasalazine BASDAI responders from non-responders in IBD-pSpA. (A-B) Mean A BASDAI between baseline and week 12 of controls compared to sulfasalazine (SAS) treatment (A) and sulfasalazine treatment stratified by IBD diagnoses. (B) Red line represents the cut off defining clinical response. Error bars represent the SEM. **P≤.01, t test. (C) Linear correlation between fecal abundance of sulfapyridine and Δ BASDAI. (D) Box plots comparing alpha diversity, based on Shannon index, between baseline and week 12. Wilcoxon matched-pairs signed-rank test P-values are shown. (E) Principal coordinate analysis plot is shown using Bray-Curtis and stratified by BASDAI clinical response and time of sample collection. Monte Carlo, PERMANOVA P values are shown. (F) Bar plot displays the differentially abundant microbial species between responders and non-responders identified by LEfSe at baseline (P<. 05, Mann-Whitney). Modules with a linear discriminant analysis (LDA) score>2 are plotted. (G) ROC curves demonstrating the ability of the indicated bacterial taxa in discriminating responders from non-responders in a separate validation cohort (n=16). AUC is indicated. *P≤.05, **P≤.01.

FIG. 2. Baseline differences in butyrate synthesis pathway stratifies sulfasalazine clinical response. (A) Principal coordinate analysis plot is shown using Bray-Curtis and stratified by BASDAI clinical response and time of sample collection. Monte Carlo, PERMANOVA P value are shown. (B) Box plots comparing median carbohydrate degradation subpathways abundance (copies per million) between responder and non-responder at baseline (see FIG. 8A, marked in bold). 1: dTDP L-rhamnose biosynthesis I; 2: Superpathway of hexuronide and hexuronate degradation; 3: D-galacturonate degradation I; 4: Superpathway of β-D-glucuronide and D-glucuronate degradation; 5: D-galactose degradation V (Leloir pathway); 6: Sucrose degradation III (sucrose invertase); 7: Glutaryl-CoA degradation; 8: Galactose degradation I (Leloir pathway); 9: Four-deoxy-L-threo-hex-4-enopyranuronate degradation; 10: Starch degradation V; 11: D-fructuronate degradation. Boxplots present the median, 25th and 75th percentiles, Mann-Whitney P<05 is shown. (C) Schematic of butyrate synthesis pathway from Glutary-CoA degradation. ccr, crotonyl-CoA reductase, gcd, glutaryl-CoA dehydrogenase; but, butyryl-CoA: acetate CoA transferase; buk, butyrate kinase. (D) Relative abundance of fecal short chain fatty acid was determined by mass spectrometry and normalized per mg of fecal sample used. T-test, * P<.05, ** P<.01 is shown. (E) Baseline but and buk mean abundances (copies per million) between responders and non-responders. Error bars represent SEM. *P<.05; t test.

FIG. 3. Sulfapyridine-induced folate stress regulates Faecalibacterium prausnitzii transcription of butyrate synthesis pathway. (A) Average percentage of but reads from the IBD-pSpA cohort at baseline. Metagenomic sequences aligned by bacterial species based on 90% homology. (B-C) F. prausnitzii grown anaerobically with sub-inhibitory dose of sulfapyridine (SP, 1000 μM) or sodium hydroxide vehicle (control) over 46 hours. (C) Principal component plot from RNA-seq analysis performed at hour 46 depicted in letter B. On average four million reads mapped to F. prausnitzii (˜10X genome coverage). (D) Schematic highlights pathway and enzymes regulating butyrate synthesis. Normalized counts (log₂ copies per millions) of genes related to the F. prausnitzii butyrate synthesis pathway genes. Error bars represent the SEM. *P<.05, ****P<.0001, DESeq (P adjusted, FDR). (E) Butyrate abundance measured by mass spec following in vitro culture of for 22 or 46 hours with either vehicle control (C) or SP treatment as indicated. Technical replicates are shown, **P<01. (F) Fold change of but transcription in either F. prausnitzii or E. rectale at 22 hours with either vehicle control (C) or SP treatment as indicated. Technical replicates are shown, *P<.05. (G) In vitro F. prausnitzii but expression was measured at hour 30 post-bacteria exposure to sulfapyridine (SP), folate and/or sodium hydroxide vehicle (control) by qPCR. Scatter plots error bars represent the SEM. ****P<.0001, ANOVA, Tukey's multiple comparison test. (H) Boxplot comparing the median deoxyuridine and thymine fecal abundance within BASDAI clinical response groups (R: responders; NR: non-responders). Boxplots present the median, 25th, and 75th percentiles; *P<.05; Wilcoxon matched-pairs signed-rank test.

FIG. 4. Sulfasalazine promotes butyrate production and limits colitis. (A) Fecal acetate, butyrate, and propionate levels of germ-free (GF) or Jackson specific pathogen free (SPF) mice at day 14 post-treatment initiation with sulfasalazine (SAS), balsalazide (BSD), or water (H₂O) control. Graphs show data from 2 independent experiments. Error bars represent SEM. **P<.01; ANOVA with Tukey's multiple comparison test. (B, C) Jackson SPF were treated with SAS or H₂O vehicle for 7 days and then exposed to 2% DSS ad libitum for 7 days. Treatments were maintained throughout the experiment. Percent survival (B), and lipocalin (C) are shown. Graph shows data from 2 independent experiments (H₂O, n=9; SAS, n=8; BSD, n=9). (D, E) SPF Gpr109a^{-/-}, and het littermate controls were treated with SAS or H₂O vehicle for 7 days and then exposed to 2% DSS ad libitum for 7 days. SAS treatment was maintained throughout the experiment. Percent survival (D), and fecal lipocalin (E) are shown. Graph shows data from 2 independent experiments (Het-H₂O, n=7; Het-SAS, n=6; Gpr109a^{-/-}−H₂O, n=10; Gpr109a^{-/-}−SAS, n=9). Survival analysis; *P<.05; **P<01; log-rank (Mantel-Cox) test. Boxplots present the median, 25th, and 75th percentiles, ANOVA with Kruskal-Wallis multiple comparison test, P values are shown.

FIG. 5. Sulfasalazine reduces colitis in gnotobiotic mice colonized with responder microbiomes or non-responder microbiomes with F. prausnitzii. (A) Schematic of gnotobiotic mouse colonization. (B, C) Fecal butyrate per mg of feces at 7 days after colonization prior to initiation of DSS. One representative of three responders (B) or non-responders (C) is shown as indicated. (D-F) Germ-free mice received fecal microbial transplants (FMT) from three responders (D) or three non-responder subjects (E,F). F. prausnitzii group was orally gavaged with F. prausniztii prior to DSS (E,F). SAS treatment was initiated on 14 days post-FMT and maintained throughout the experiment. Mice were exposed to 2% DSS ad libitum for 7 days starting 7 days post-SAS initiation. Weight loss and levels of lipocalin in cecal contents are shown (E, F). Graphs show average data (thick line) of three individual donors (thin line). Each thin line is average of n=3-5 mice/donor. Dark lines are average and SEM of pooled data from all three donors. Total responder-H₂O, n=12; responder-SAS, n=14; non-responder-H₂O, n=10; non-responder-SAS, n=13; non-responder+F. prausnitzii=9 non-responder-SAS+F.prausnitzii, n=9). Mixed-effects model (weight loss) or Kruskal-Wallis test with multiple comparison (lipocalin). P values are shown. Error bars represent SEM; *P <.05, **P<.01, ****P<.0001.

FIG. 6. Clinical SpA response to SAS correlates with changes in the gut microbiome (Related to FIG. 1). (A) Mean Δ ASDAS, HBI and CRP between baseline and week 12 of controls compared to sulfasalazine (SAS) treatment. (B) Mean Δ BASDAI between baseline and week 12 comparison between SAS doses. Mean comparison of (C) Δ BASDAI between subjects current receiving (yes) or not receiving (no) biologic agents or mesalamine therapy at stable doses. Red line represents the cut off for clinical disease improvement. Error bars represent SEM. *P<.05; t test. (D) Box plot illustrating SAS and sulfapyridine (SP) fecal levels at baseline and week 12. Boxplots present the median, 25th and 75th percentiles. (E) Linear correlation between fecal abundance of 5-aminosalycilic acid and Δ BASDAI. (F) Linear regression analysis between Δ Shannon index and Δ BASDAI between baseline and week 12 for all subjects. R2 and P values are shown.

FIG. 7. Bacterial taxa enriched in SAS responder microbiome at baseline (Related to FIG. 1). (A) Heatmap depicting the relative abundance of the six bacterial species identified by LEfSe in responders and non-responders at baseline (P<.05, Mann-Whitney). (B-C) Boxplot comparing the median relative abundance of the combined six microbial markers or F. prausnitzii between responders and nonresponders at baseline in the discovery cohort (B) or the validation cohort (C). **P<.01; *P<.05 Mann-Whitney. (D) Receiver operating characteristics (ROC) were generated for all 6 bacterial markers identified in the initial cohort. F. prausnitzii had the highest AUC of all individual taxa and the combination of F. prausnitzii+R. callidus achieved the highest AUC of all combinations.

FIG. 8. Metagenomic pathway analysis identifies butyrate synthesis pathway enrichment in responders (Related to FIG. 2). (A) Bar plot displays the 30differentially abundant microbial pathways between responders and non-responders identified by LEfSe at baseline (P<0.05, Mann-Whitney). Modules with a linear discriminant analysis (LDA) score>2 are plotted. Pathways belonging to carbohydrate degradation superpathways are marked in bold. (B) Microbial contribution to the carbohydrate degradation pathways marked in bold above. Average percentage of pathways reads by bacterial species based on 90% homology. 1: dTDP Lrhamnose biosynthesis I; 2: Superpathway of hexuronide and hexuronate degradation; 3: D-galacturonate degradation I; 4: Superpathway of β-D-glucuronide and D-glucuronate degradation; 5: D-galactose degradation V (Leloir pathway); 6: Sucrose degradation III (sucrose invertase); 7: Glutaryl-CoA degradation; 8: Galactose degradation I (Leloir pathway); 9: Four-deoxy-L-threo-hex-4-enopyranuronate degradation; 10: Starch degradation V; 11: D-fructuronate degradation.

FIG. 9. Sulfapyridine-induced folate stress regulates Faecalibacterium prausnitzii (Related to FIG. 3). (A) CFU counts for F. prausnitzii grown anaerobically in YCFAC media containing 500, 1000 or 2000 μM of sulfapyridine (SP) or NaOH vehicle (control) for 26 h. Error bars represent SEM. Each dot represents a technical replicate from two independent experiments. (B) In vitro E. rectale growth anaerobically with sulfapyridine (SP, 1000 μM) or sodium hydroxide vehicle control (NaOH), folate (566 μM). (C) Normalized counts (log2 copies per millions) of F. prausnitzii folate stress related genes. Scatter plots error bars represent the SEM. *P<.05, ****P<.0001, DESeq (P adjusted, FDR). (D) Drug action and folate trap schematic. De novo thymidine diphosphate synthesis is marked in green and the salvage pathway is marked in orange. PABA, para-aminobenzoate; DHPS, dihydropteroate synthase; THF, tetrahydrofolate; dUMP, dioxyuridine monophosphate; dUTP, dioxyuridine triphosphate; TMP, thymidine monophosphate; TDP, thymidine diphosphate; TTP, thymidine triphosphate. (E, F) Normalized counts (log2 copies per millions) of Tdk gene expression described in main FIGS. 3B-E (E) and thymine target metabolomics analysis from F. prausnitzii cultures (F) also described on main FIGS. 3B-E Error bars represent SEM. *P<.05, t test. (G) In vitro F. prausnitzii growth anaerobically with sulfapyridine (SP, 1000 μM) or sodium hydroxide vehicle control (NaOH), folate (566 μM) or folate+sulfapyridine (folate=566 μM, SP=1000 μM).

FIG. 10. Sulfasalazine promotes butyrate production and limits colitis in SPF and gnotobiotic mice colonized with a responder microbiome (Related to FIGS. 4 and 5). (A-B) Fecal metagenomic analysis of WT mice from Jackson labs. (A) Baseline but and buk mean abundances (copies per million) and but and buk abundances at day 14 post-treatment between sulfasalazine (SAS) and H2O vehicle control. Error bars represent SEM. ***P<.001; t test. (B) Fecal metabolomics analysis of WT mice described in main FIGS. 4F-G. Sample were collected at day 7 post-treatment initiation, at the day of DSS initiation. Bar plot illustrating 5-ASA fecal levels within each treatment group evaluated (H2O vehicle control, SAS, and balsalazide (BSD)). Error bars represent SEM. (C) Jackson SPF were treated with SAS or H2O vehicle for 7 days and then exposed to 2% DSS ad libitum for 7 days. Treatments were maintained throughout the experiment. Weight loss is shown. Graph shows data from 2independent experiments (H2O, n=9; SAS, n=8; BSD, n=9). (D) SPF Gpr109a−/−, and het littermate controls were treated with SAS or H2O vehicle for 7 days and then exposed to 2% DSS ad libitum for 7 days. SAS treatment was maintained throughout the experiment. Weight loss is shown. Graph shows data from 2 independent experiments (Het-H2O, n=7; Het-SAS, n=6; Gpr109a−/−−H2O, n=10; Gpr109a−/−−SAS, n=9). (E) Principal coordinate analysis plot is shown using Bray-Curtis for donor sample (triangle) and fecal sample of mouse recipient (circle) from an independent donor (labeled by color). PERMANOVA p value 0.001 is used to show significant clustering of donor and recipient microbiomes. (F) Mice were exposed to 2% DSS ad libitum for 7 days starting from day 7 post-SAS initiation. Levels of lipocalin in cecal contents are shown. Boxplot present the median, 25th, and 75th percentiles; ANOVA, Kruskal-Wallis test P value is shown.

FIG. 11 provides Supplemental Table 1.

FIG. 12 provides Supplemental Table 2.

### DETAILED DESCRIPTION

Unless defined otherwise, all technical and scientific terms used herein have the same meaning as commonly understood by one of ordinary skill in the art to which this invention belongs.

Abbreviations used in this disclosure include: ASV, amplicon sequence variant; AUC, area under the curve; ASDAS, Ankylosing Spondylitis Disease Activity Score; ASAS, Assessment of Spondyloarthritis International Society; BASDAI, Bath Ankylosing Spondylitis Disease Activity Index; CD, Crohn's disease; CPM, copies per million; CRP, C-reactive protein; CV, cross-validation; DHPS, dihydropteroate synthase; DSS, dextran sulfate sodium; dUMP, dioxyuridine monophosphate; dUTP, dioxyuridine triphosphate; ESR, erythrocyte sedimentations rate; FMT, fecal microbial transplantation; GF, germ-free; HBI, Harvey-Bradshaw Index; IBD, inflammatory bowel disease; LDA, linear discriminant analysis; LEfSe, linear discriminant analysis effect size; LOO-CV, leave-one-out-cross-validation; PABA, para-aminobenzoate; PCoA, principal coordinate analysis; ROC, receiver operating characteristic; SOC, standard of care; pSpA, peripheral spondyloarthritis; SpA, spondyloarthritis; SP, sulfapyridine; SAS, sulfasalazine; THF, tetrahydrofolate; TMP, thymidine monophosphate; TDP, thymidine diphosphate; TTP, thymidine triphosphate; UC, ulcerative colitis.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another example includes from the one particular value and/or to the other particular value. Similarly, when values are expressed as approximations, by the use of the antecedent “about” it will be understood that the particular value forms another example. The term “about” in relation to a numerical value encompasses variations of +/−10%, +/−5%, or +/−1%.

The disclosure provides improved approaches to treating IBD by identifying a subset of IBD patients for whom administration of SAS and bacteria results in enhanced efficacy of the SAS treatment. In examples, the individual has been diagnosed with a form of IBD and spondyloarthritis. In examples, a biological sample from the individual who has been diagnosed with IBD and spondyloarthritis is or has been analyzed by one or more described approaches, prior to a described treatment.

In an example, a gastrointestinal tract and/or a fecal sample is used to produce a fecal biome genetic sequence. At least in part by using bacterial biome sequence information, the disclosure provides a method for selecting patients who have non-SAS responder microbiomes. Such patients are then administered a combination of SAS and bacteria to thereby enhance one or more therapeutic effects of the SAS. The disclosure accordingly includes use of SAS in combination with all single bacteria types that that exhibit a described enhanced effect of SAS treatment, and all combinations of bacteria types that are shown or can be expected from the present disclosure to exhibit the described enhancement of SAS treatment.

In examples the bacteria isolated and tested from an individual to determine if the individual is a candidate for treatment with a described combination therapy may be characterized by downregulation of the folP-like enzyme (MBL-fold metallohydrolase). In examples, the bacteria isolated and tested to determine if the individual is a candidate for treatment with a described combination therapy may be characterized by upregulation in folC (dihydrofolate reductase) and folT (folate ECF transporter S component FolT). In an example, the bacteria isolated and tested from an individual to determine if the individual is a candidate for treatment with a described combination therapy exhibit consumption of thymine via the salvage pathway with subsequent accumulation of deoxyuridine. Accordingly, and without intending to be bound by any particular theory, the foregoing gene expression patterns may be considered to characterize the bacteria used in methods of this disclosure as comprising a functional “folate trap.” Thus, use of one or more described of the described bacteria may be considered a means of introducing a folate trap into the individual who is also treated with SAS to thereby enhance the therapeutic effect of SAS on a selected individual.

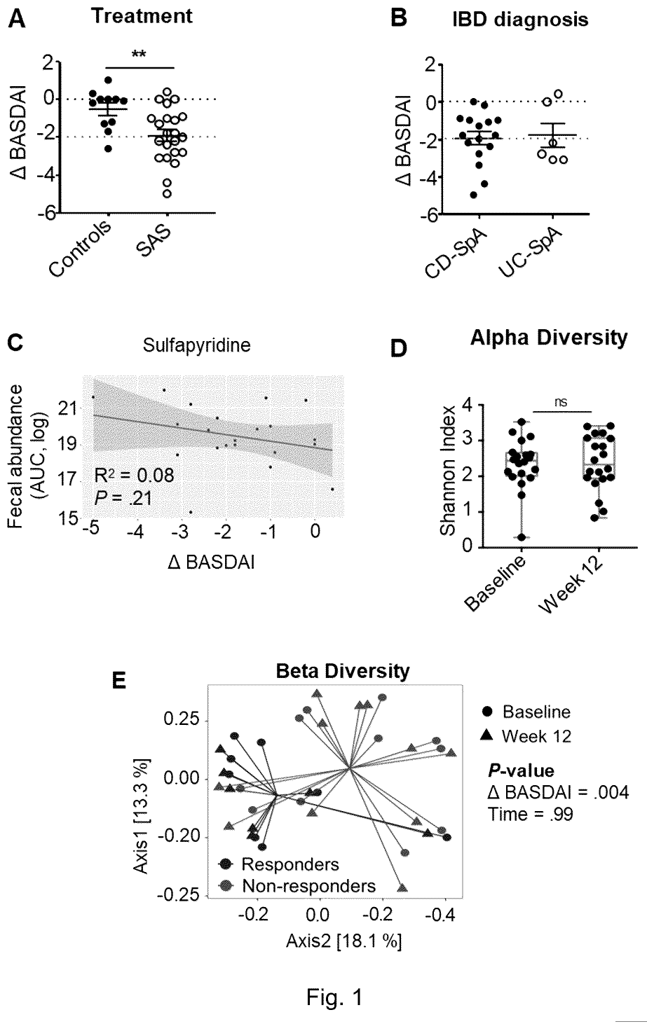

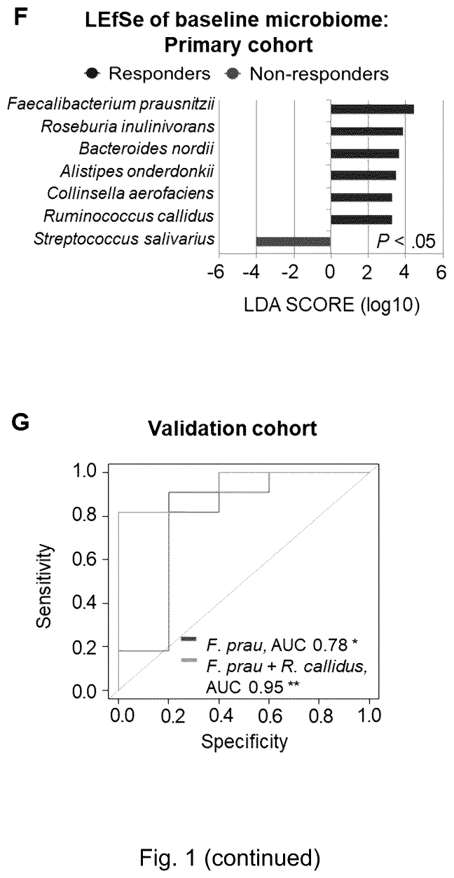

The relative expression of any gene described herein, and the presence, absence, and/or amount of any bacteria in a biological sample can be determined using routine techniques when given the benefit of the present disclosure. In general, a value determined to select an individual to receive a described combination therapy can be compared to a suitable reference value that can be derived from a series of individuals who have been determined to have IBD and optionally spondyloarthritis but are SAS responders without having received an exogenously provided bacteria preparation as described herein. Thus, in examples, a biological sample analyzed according to a described method and used to select an individual for a described combination treatment may be compared to a reference value, such as any suitable control. In examples, the reference value is a numerical threshold, any statistical value, a receiver operating characteristic (ROC), an area under a curve (AUC), a P value, or any value obtained from repeated measurements taken from IBS patients who are SAS responders and who are determined to be SAS responders without manipulating their gastrointestinal genomes. In examples, to discriminate responders vs. non-responders F. prausnitzii may have an AUC of approximately 0.78. In an example, a method of the disclosure may use a combination of all the taxa identified (FIG. 1G). Combining F. pransnitzii with R. callidus may have an AUC of approximately 0.95. In an example, an SAS non-responder has Streptococcus salivarius in a biological sample obtained from the individual. In examples, discriminating SAS responders from non-responders can include analysis the expression of any one or combination of genes as described herein.

In examples, only 1, only 2, only 3, only 4, only 5, only 6, or only 7 different types of bacteria are administered to an individual. In examples, the bacteria administered to the individual include at least one of F. prausnitzii, Ruminococcus Callidus, Alistipes onderdonkii, Bacteroides nordii, Collinsella aerofaciens, or Roseburia imulinivorans. In an example, at least or only F. prausnitzii is administered to the individual, in combination with SAS. In an example, a combination consisting of any combination of the described bacteria is administered. In an example, a combination consisting of Combining F. prausnitzii and R. Callidus is administered.

Bacteria for use in the described methods can be obtained from any suitable source, such as from the American Type Culture Collection (ATCC), or may be isolated from a responder patient and used directly, or after culturing. The disclosure is not necessarily limited to any particular strain of the described bacteria.

In an example the disclosure includes a supplement product, such as a nutraceutical product, or a dietary supplement, or a food ingredient, including but not limited to a probiotic formulations or functionals food that contains one or more live bacteria as described herein. The supplement product can be provided in the form of, for example, a liquid, capsules, tablets, softgels, powders, freeze-dried compositions, and the like. In examples, the supplement product may be provided as enteric-coated capsules.

In examples, administration of the described bacteria may be performed such that the bacteria are introduced into the gastrointestinal system. In examples, the administration can be effected by enema or colonoscope, or via intubation of the small bowel using for example a large bore catheter equipped with a distal balloon to effect rapid passage down the jejunum.

The parameters for use of SAS are known in the art and can be adapted with the presently described method to provide a therapeutically effective amount of SAS, although the amount of SAS used may be lower than previously described approaches due to the enhancement of SAS efficacy when used in combination with the described bacteria.

The term “therapeutically effective amount” as used herein refers to an amount of SAS and the described bacteria sufficient to achieve, in a single or multiple doses, the intended purpose of treatment. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of bacteria and SAS to achieve a desired effect. Additional factors which may be taken into account include the type of IBD (i.e., the degree of inflammation in the digestive tract, and/or whether the IBD is ulcerative colitis affecting the large intestine and/or colon, with or without concomitant inflammation and ulcers in the lining of the colon, or Crohn's disease, which can affect any part of the gastrointestinal tract from the mouth to the anus. Additional factors that can be taken into account include the weight and gender of the patient, desired duration of treatment, method of administration, time and frequency of administration, drug combination(s), reaction sensitivities, and tolerance/response to therapy. In certain examples, a therapeutically effective amount is an amount that reduces one or more signs or symptoms of a disease, and/or reduces the severity of the disease. A therapeutically effective amount may also inhibit or prevent the onset of a disease, or a disease relapse. In examples, performing a described method improves a BASDAI and/or ASDAS value.

In example, a therapeutically effective amount of described bacteria are administered. In examples, a therapeutically effective amount comprises 1 million to 100 billion colony forming units, inclusive, and include all numbers and ranges of numbers there between. In examples, the SAS and described bacteria are administered concurrently or sequentially. In examples, the bacteria are administered first, followed by SAS administration. In examples, the SAS is administered first, followed by bacteria administration.

The disclosure includes the following non-limiting aspects.

A method to treat a subject with IBD and spondyloarthritis comprising: assaying the subject's microbiome for evidence of a functional folate trap and administering sulfasalazine to subjects with a functional microbiome folate trap, or administering sulfasalazine in combination with described bacteria comprising a functional folate trap to subjects lacking a functional folate trap. In an example, the presence of a functional microbiome folate trap is assessed by measuring relative abundance of F. prausnitzii in the microbiome with shotgun metagenomic sequencing or 16S based PCR wherein a relative abundance of F. prausnitzii between 0.5% to 3.5%, between 1% and 3%, or 2%, indicates a functional microbiome folate trap. In an example, the presence of Ruminococcus callidus indicates a functional microbiome folate trap. In an example, the administered folate trap comprises at least one of Alistipes onderdonkii, Bacteroides nordii, Collinsella aerofaciens, F. prausnitzii, Roseburia imilinivorans or Ruminococcus Callidus. In an example the disclosure includes a method to treat a subject with IBD and spondyloarthritis comprising: measuring baseline thymine fecal levels or baseline fecal deoxyuridine levels prior to administering sulfasalazine to the subject; administering sulfasalazine to the subject; measuring whether the subject responded to sulfasalazine; measuring fecal thymine levels or fecal deoxyuridine levels after administering sulfasalazine to non-responding subjects; administering a folate trap to non-responding subjects if fecal deoxyuridine in the second measurement was at least 150% of the baseline measurement or fecal thymine is less than 60% of the baseline.

The following Examples are intended to illustrate but not limit the disclosure.

### Example 1

### BASDAI Identifies Clinical Response in Joint Symptoms to SAS in IBD-pSpA

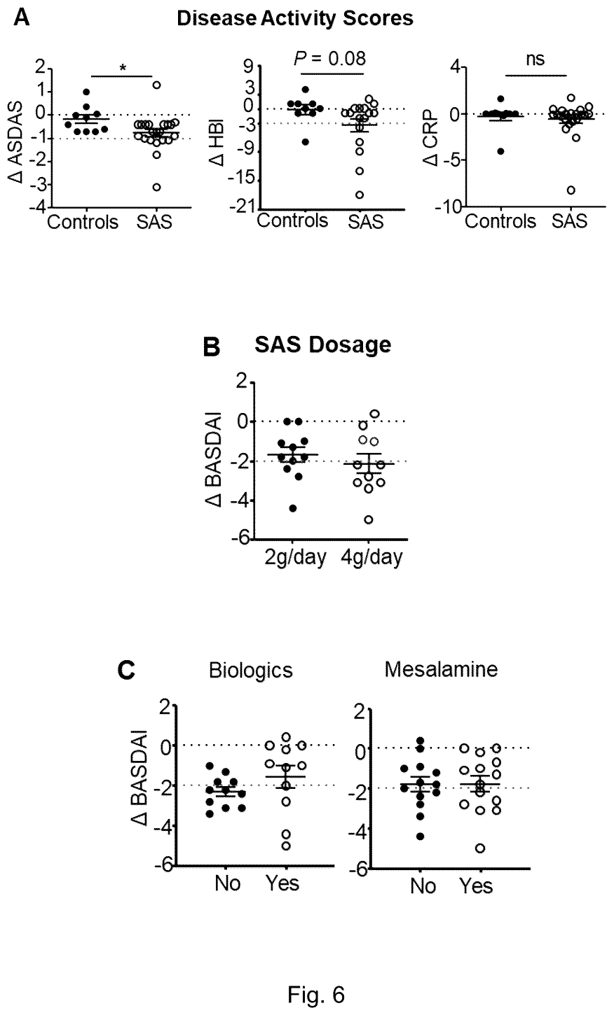

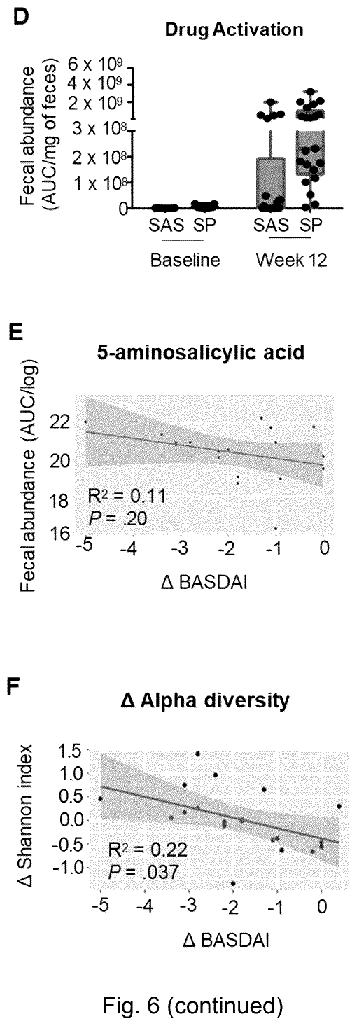

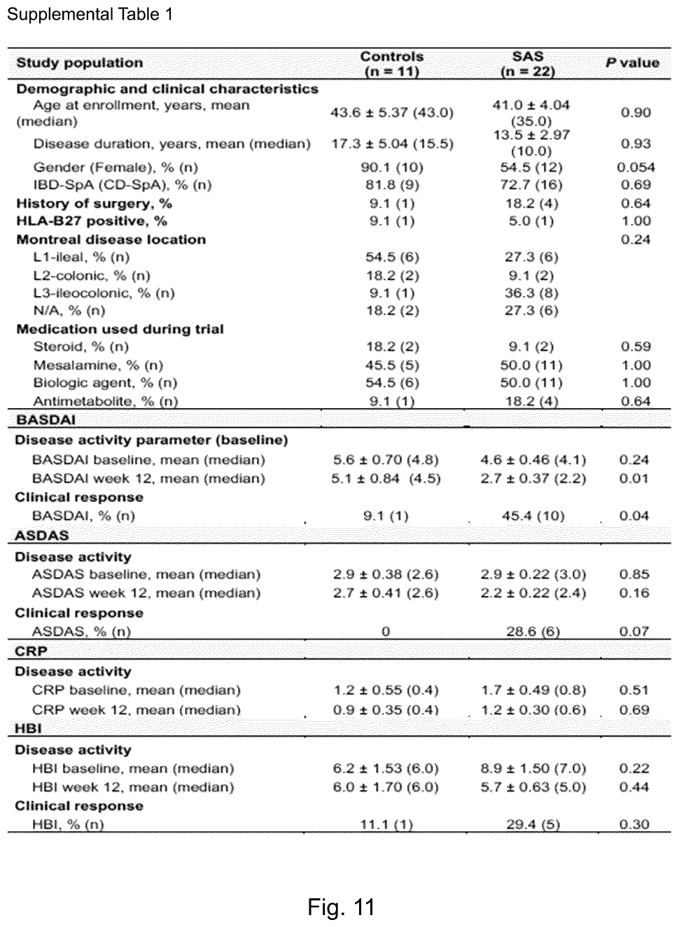

Thirty-three sequential patients with a clinical diagnosis of IBD and ASAS-defined SpA were enrolled (Supplementary Table 1/FIG. 11); CD 78.0%, n=25; UC 20.0%, n=8). Twenty-two of the enrolled patients were treated with SAS therapy while 11, who were either intolerant or refused SAS therapy, were followed as standard of care controls. No significant differences were found between treatment groups in terms of IBD diagnosis, age, disease duration, Montreal disease locations, HLA-B27 status or history of surgery; however, the proportion of females enrolled was higher in controls compared to SAS treated patients (P=.054, Supplementary Table 1/FIG. 11). A significantly higher proportion of patients achieved clinical response in joint symptoms (defined by a reduction in BASDAI of >2) at week 12 in the SAS group compared to controls (45.4% vs. 9.1%, P=.04, Table S1). Participants treated with SAS also had a significant absolute score reduction in BASDAI compared to controls (FIG. 1A). A similar trend in clinical improvement was observed using ASDAS (28.6% vs. 0%, P=.07,Table S1/FIG. 11; FIG. 6A), but no difference in CRP alone was observed (Table S1/FIG. 11 and FIG. 6A). Consistent with concordance between intestinal and peripheral SpA symptoms, a higher proportion of patients receiving SAS had clinical improvement in the Harvey-Bradshaw Index (HBI) compared to standard of care controls (29.4% vs. 11.1%, P=.30; FIG. 6A, P=.08). The absolute reduction in BASDAI for subjects treated with SAS was similar between CD and UC (FIG. 1B). Of the 22 patients treated with SAS, 50% (n=11) tolerated increase to 4 mg/day; however, participants taking 2 mg or 4 mg daily had equivalent reduction in all joint disease activity scores (FIG. 6B). Baseline biologic or mesalamine therapy did not impact the change in disease activity scores following SAS treatment (FIG. 6C).

### Example 2

### The gut Microbiome Stratifies IBD-pSpA Subjects With Clinical Response to SAS

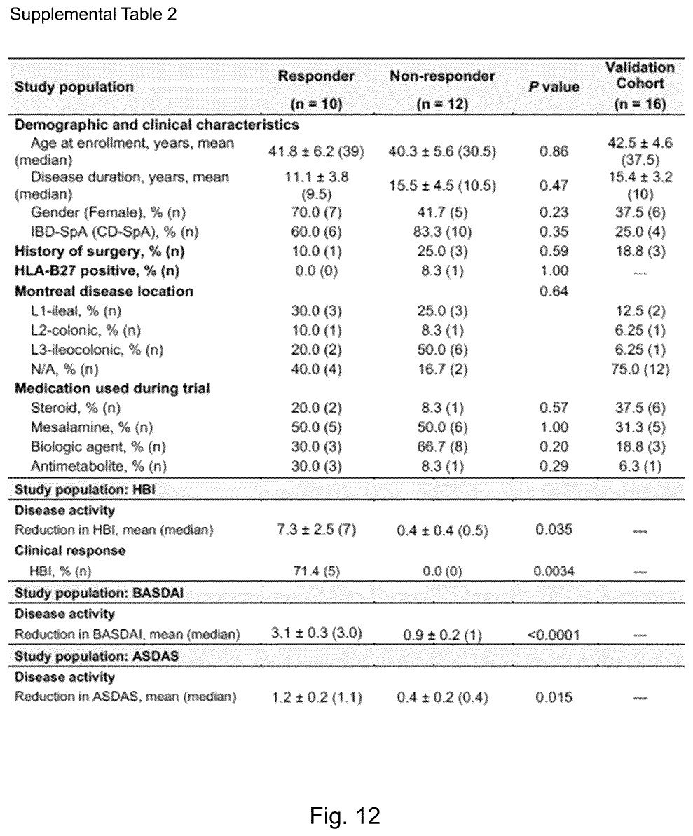

Given the higher proportion of IBD-pSpA subjects treated with SAS showing clinical response by BASDAI, the disclosure includes identification of biomarkers associated with this effect. No significant differences were found between SAS responders and no-responders in terms of IBD diagnosis, age, disease duration, Montreal disease locations, HLA-B27 status or history of surgery (Table S2/FIG. 12). In addition to significant reduction of BASDAI and ASDAS in responders, a concordant reduction was also seen in intestinal symptoms measured by HBI.

Since SAS is a prodrug, we performed fecal metabolomics to assess intestinal drug conversion. SAS and its cleavage product SP were detected in week 12 fecal samples (FIG. 6D). SAS and/or SP were detected in all samples, but the variability in fecal SP may reflect less efficient conversion of prodrug or clearance. To evaluate whether SAS conversion to SP associates with pSpA clinical response, we performed linear regression analysis. There was no significant correlation between SP or 5-ASA fecal levels and A BASDAI (FIG. 1C and FIG. 6E).

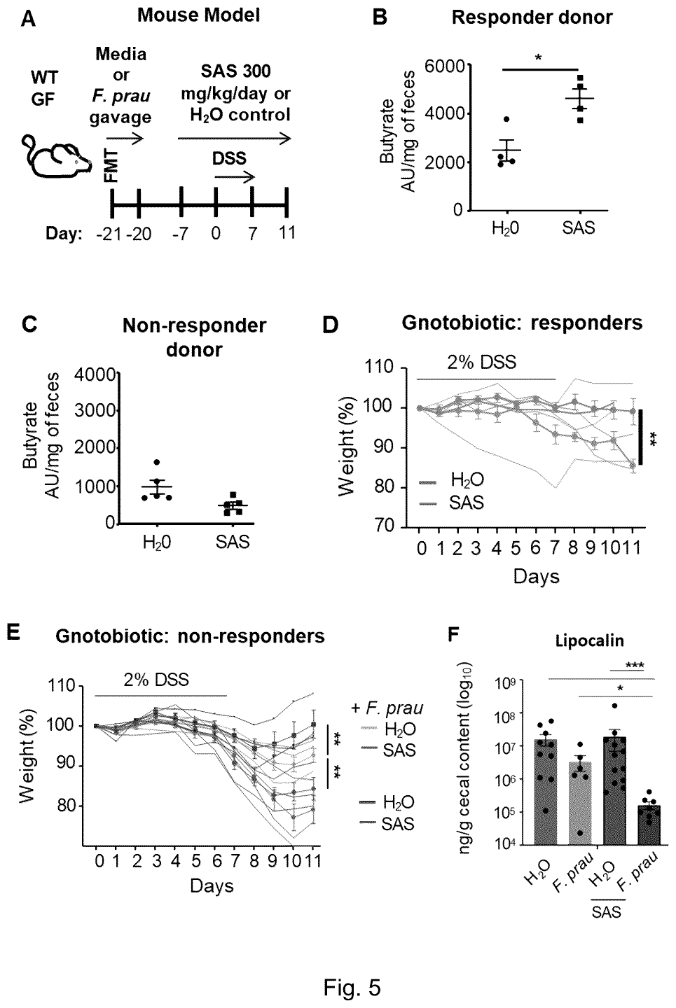

With the potential role for SP in regulating the gut bacteria, we next sought to evaluate the possibility that the clinical response of SpA symptoms to SAS depends on the diversity of the gut microbiome using 16S rRNA sequencing. Although no significant differences in alpha diversity, as measured by Shannon index, were observed between baseline and week 12 (FIG. 1D), increase in alpha diversity correlated with improvement in BASDAI (FIG. 5F, P=0.037).

To determine the microbiome composition and its functional potential, we performed metagenomic sequencing (average reads/sample ˜4.0×10⁶, SEM±3.7×10⁵ at baseline and 4.6×10⁶±3.3×10⁵ at week 12). To evaluate whether the microbial community was affected by SAS treatment, principal coordinate analysis (PCoA) based on the Bray-Curtis distance derived from the taxa relative abundance was performed. Although PCoA revealed no differences in the overall microbial structure between baseline and week 12, significant differences were found between the microbial composition of SAS responders compared to non-responders (FIG. 1E).

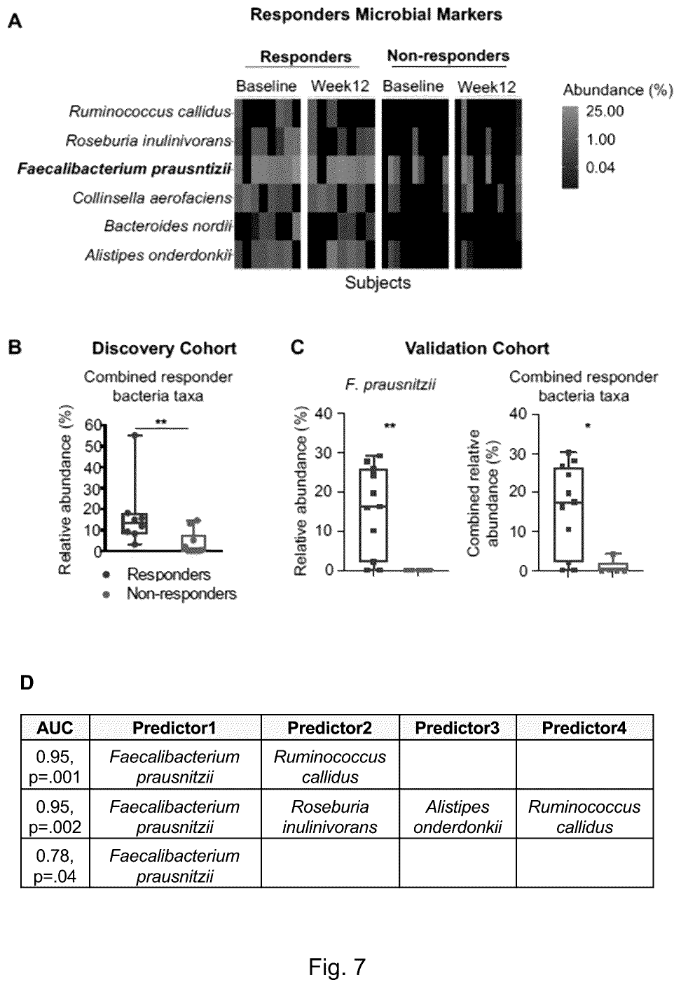

We next sought to determine the baseline taxa associated with clinical response to SAS by using linear discriminant analysis (LDA) effect size (LEfSe)³¹. Six bacterial species, including Alistipes onderdonkii, Bacteroides nordii, Collinsella aerofaciens, F. prausnitzii, Roseburia imulinivorans and Ruminococcus callidus were found to be significantly enriched in clinical responders while Streptococcus salivarius was enriched in non-responders (FIG. 1F, Mann-Whitney<.05; FIGS. 7A and B). Microbial markers were stably enriched in responders at week 12 (FIG. 6A).

To validate these bacterial taxa as potential markers of sulfasalazine response, we generated a validation cohort using a separate biobank established at Weill Cornell Medicine. We identified 16 subjects with a diagnosis of IBD and SpA that were currently taking sulfasalazine (Table S2). Five subjects had active SpA symptoms and were characterized as non-responders, while 11 subjects had inactive SpA symptoms and were characterized as responders. We performed 16S rRNA sequencing of fecal samples from these subjects. Like the discovery cohort, the relative abundance of both F. prausnitzii and the combined bacterial taxa were higher in responders compared to non-responders (FIGS. 7B&C). We fit logistic regression models and generated ROC curves to determine the ability of the relative abundances of our marker taxa (F. prausnitzii alone or F. prausnitzii+additional taxa) to discriminate responders vs. non-responders in this separate cohort. F. prausnitzii had the highest AUC (0.78) of all the taxa identified (FIG. 1G). Combining F. prausnitzii with R. callidus (also identified in the initial cohort enriched in responders) yield ROC curves with an AUC of 0.95 (FIG. 7D).

### Example 3

### Baseline Differences in Metabolic Pathways and Butyrate Synthesis Stratifies SAS Responders

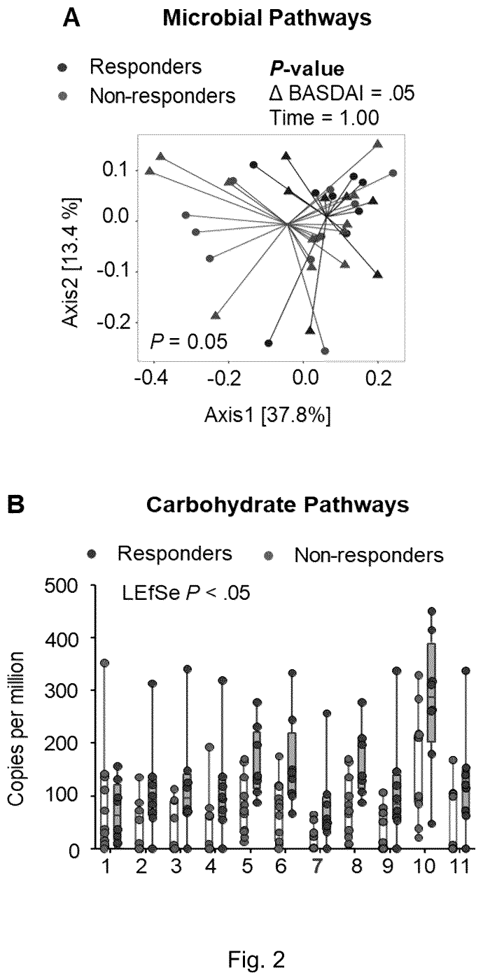

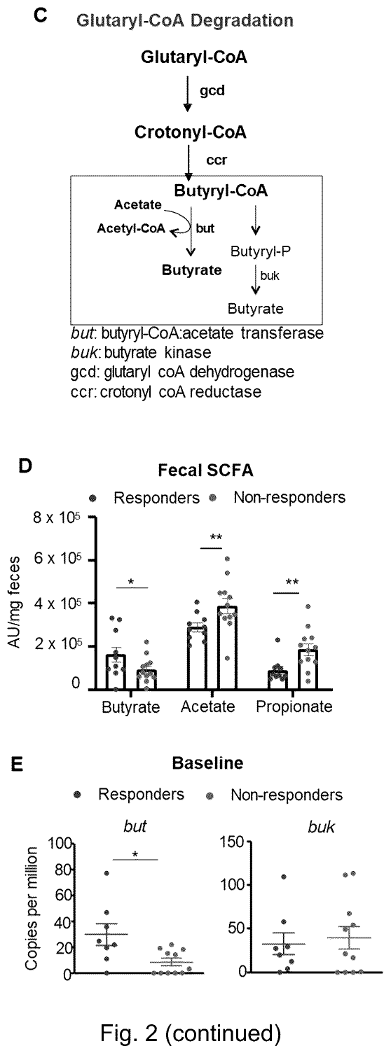

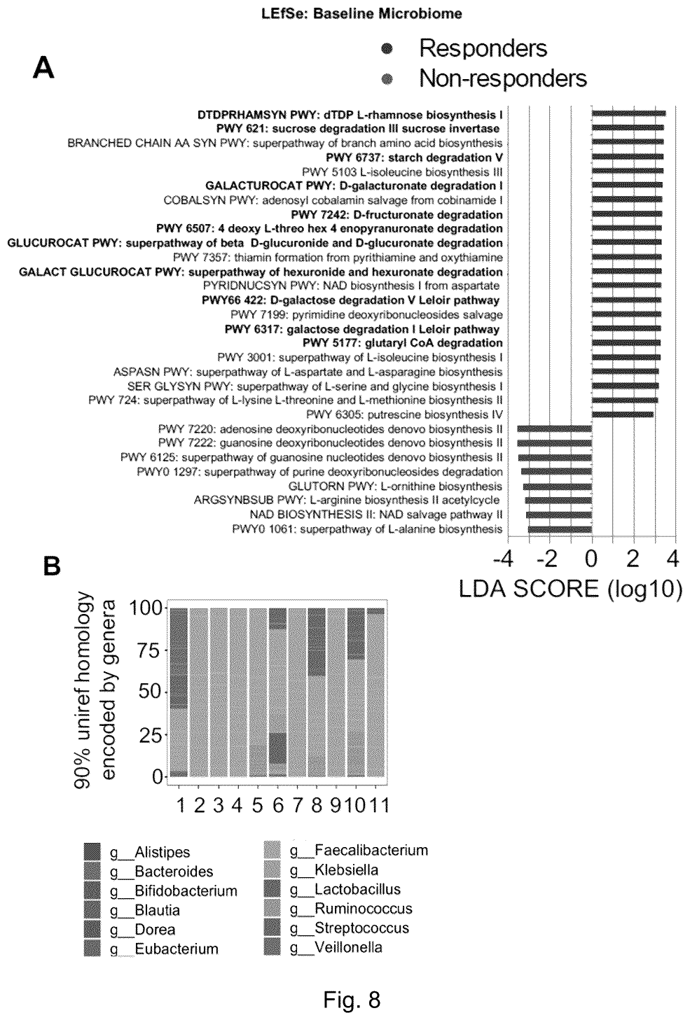

The significant differences in baseline microbiome composition between responder and non-responders supported a potential functional role for the microbiome in response to SAS. To understand the functional potential of the SAS-responsive microbiome, we performed microbial pathway analysis. PCoA based on the Bray-Curtis distance derived from the abundance (counts per million, CPM) of microbial pathways revealed significant differences between responders and non-responders (FIG. 2A). LEfSe analysis identified 30 microbial pathways differentially abundant between clinical response groups at baseline, of which, 22 were enriched in responders (FIG. 8A, Mann-Whitney<.05). The pathways enriched in responders mainly reflected sub-pathways related to basic cellular processes such as carbohydrate degradation (FIG. 2B and FIG. 8A: 50%, n=11; marked in bold), which promotes short chain fatty acid production (SCFA) characteristic of a healthy microbiome³². The majority of these differences mapped to the butyrate-producer F. prausnitzii (FIG. 8B). Specifically, the glutaryl-CoA degradation pathway (identified by LEfSe as one of the main pathways enriched in responders, pathway 7, FIG. 2B) is a main producer of butyryl-CoA leading to the production of butyrate via butyryl-CoA (FIG. 2C)^{33, 34}.

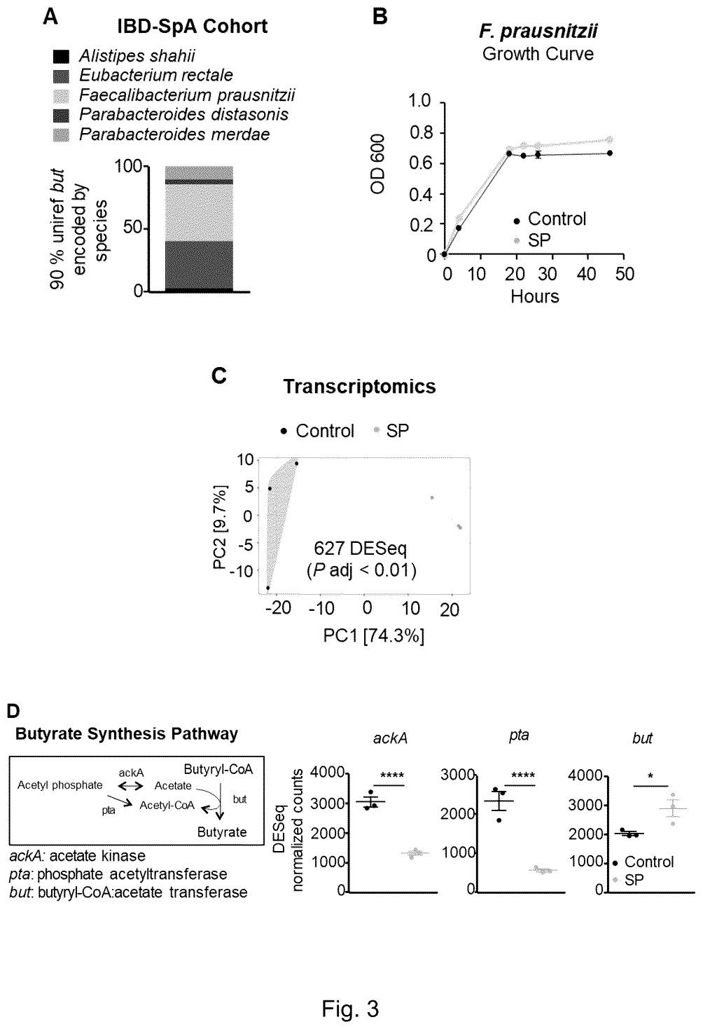

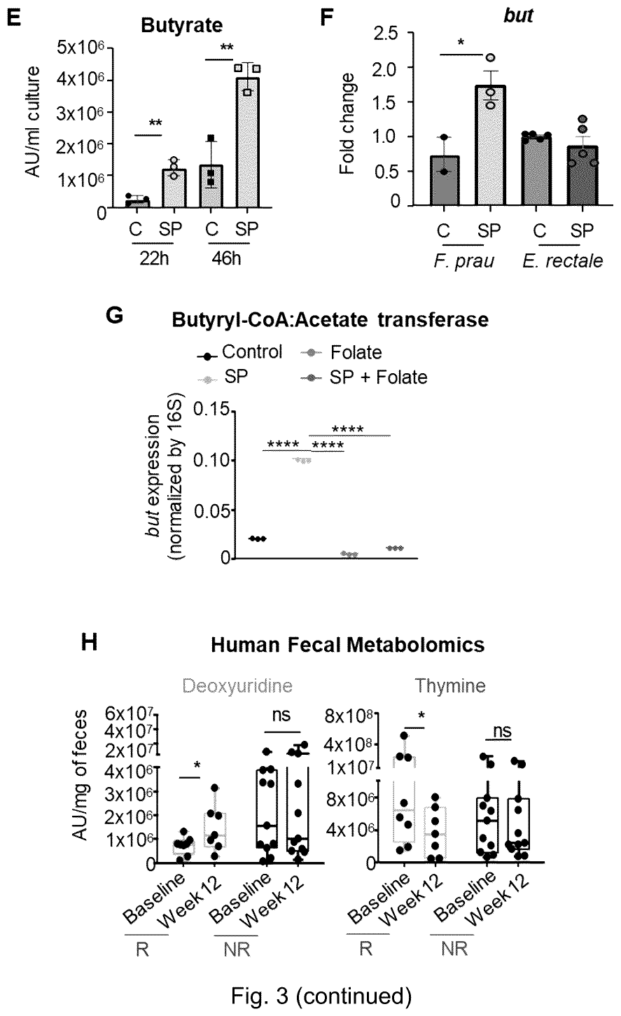

To determine if butyrate production was associated with SAS clinical response, we determined fecal SCFA concentrations for acetate, butyrate, and propionate. Consistent with the metagenomic analysis, baseline responders had higher levels of fecal butyrate and lower levels of acetate and propionate compared to non-responders (FIG. 2D). While both butyryl-CoA: acetate CoA-transferase (but) and butyrate kinase (buk) can catalyze this reaction, only but gene abundance was higher in responders compared to non-responders (FIG. 2E). At 90% similarity by Uniref, but gene mapped to F. prausnitzii, Parabacteroides merdae, Parabacteroides distasonis, Eubacterium rectale and Alistipes shahii (FIG. 3A), but only F. prausnitzii was part of the taxa differentiating responders from non-responders.

### Example 4

### SP Regulates Butyrate Synthesis and Correlates With a Folate Trap Seen in SAS Responders

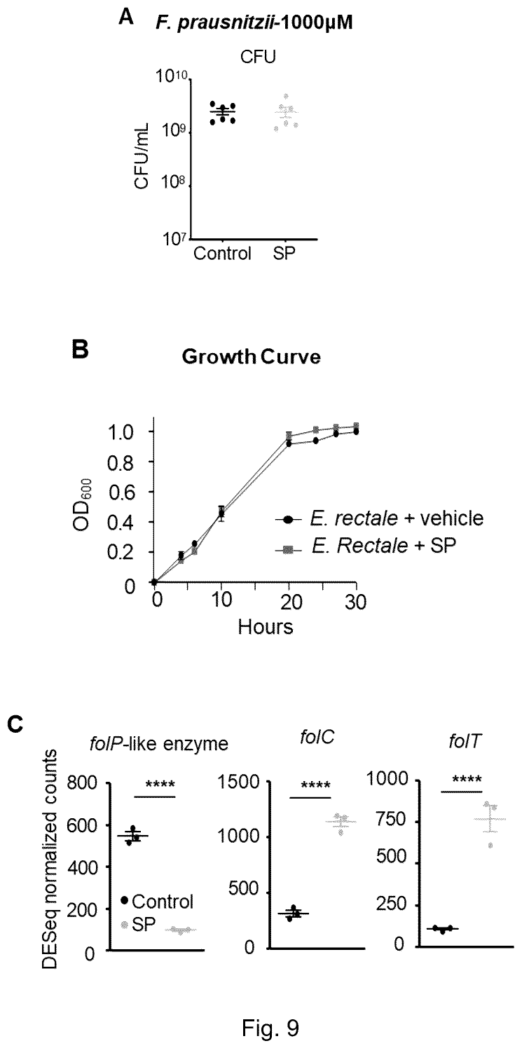

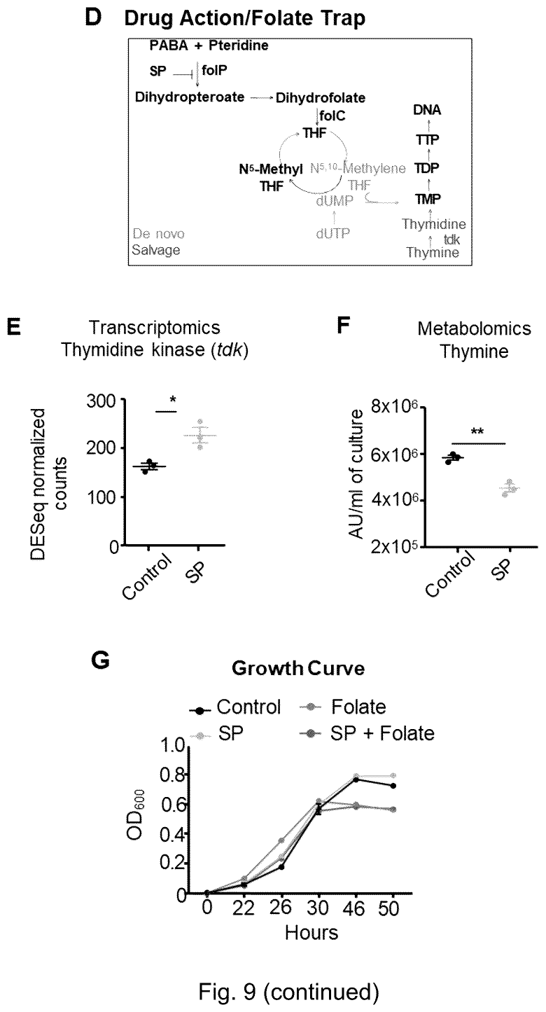

We next tested if SAS was able to modulate this butyrate synthesis pathway in bacteria. Using the bacterial taxa F. prausnitzii associated with SAS response as a candidate target, we performed bacterial RNA-seq at a sub-inhibitory SP dose of 1 mM, which is within the physiologic range of sulfasalazine in bile excretion (35-1032 ug/mL)³⁵ (FIGS. 3B, 9A). Differential expression analysis of vehicle control (NaOH) and SP treated F. prausnitzii revealed significant changes in F. prausnitzii transcription profile upon SP exposure (FIG. 3C). The most significant transcriptional targets of SP exposure included downregulation of acetate kinase (ackA) and phosphate acetyltransferase (pta), along with a concomitant increase in but expression (FIG. 3D). To test the impact of SP on the metabolic production of butyrate in complete media, mass spectrometry was performed after 22 and 46 hours of F. prausnitziiculture treated with SP or vehicle control. At both timepoints, SP treatment robustly increased butyrate production (FIG. 3E). To evaluate the specificity of SP on F. prausnitzii, we tested the impact of SP on but expression in E. rectale, the other major contributor to but expression in the microbiome. In contrast to F. prausnitzii, SP treatment at 1 mM did not change expression of but in E. rectale (FIG. 3F, FIG. 9B).

Given the ability of SP to inhibit dihydropteroate synthase (folP), we analyzed whether folate related stress contributes to the transcriptional regulation by SP observed in the F. prausnitzii in vitro assay. In our RNA-seq analysis, we detected a significant downregulation of the folP-like enzyme, MBL-fold metallohydrolase³⁶. In addition, we also observed upregulation in folC (dihydrofolate reductase) and folT (folate ECF transporter S component FolT) genes consistent with folP inhibition (FIG. 9C). During reduced availability of folate³⁷, thymine is consumed by the salvage pathway and deoxyuridine subsequently accumulates in bacterial cells (i.e. “folate trap”, FIG. 9D). Consistent with this physiology, bacterial cultures showed a significant increase in thymidine kinase expression and a corresponding decrease in thymine metabolites (FIGS. 9E, F).

To directly test the regulation of but gene by folate deficiency, F. prausnitzii was cultured with SP or NaOH vehicle control in the absence or presence of supplemental folate, using a dose previously reported to rescue bacteria from antifolate drug treatment³⁸ (FIG. 9G). Consistent with a mechanistic role for folate deficiency, the induction of but in response to SP was abrogated by supplementation with folate (FIG. 3G). Finally, to test if metabolic evidence of a folate trap correlated with clinical response to SAS, we performed metabolomics analysis on fecal samples from our original cohort. Although all subjects in this study received folate supplementation, dietary folate is primarily absorbed in the small intestine while SAS prodrug cleavage by microbial azoreductases occurs locally in the colon³⁹, enabling the potential for local folate depletion by SP in the colon. Consistent with this possibility and the folate trap effect in the F. prausnitzii in vitro model, thymine fecal abundance significantly reduced after SAS treatment in responders (P=.016), but not in non-responders (FIG. 3H). A concomitant increase in fecal deoxyuridine levels was also observed in responders (P=.05), but not in non-responders.

### Example 5

### SAS-Induced Butyrate Reduces Colitis Severity

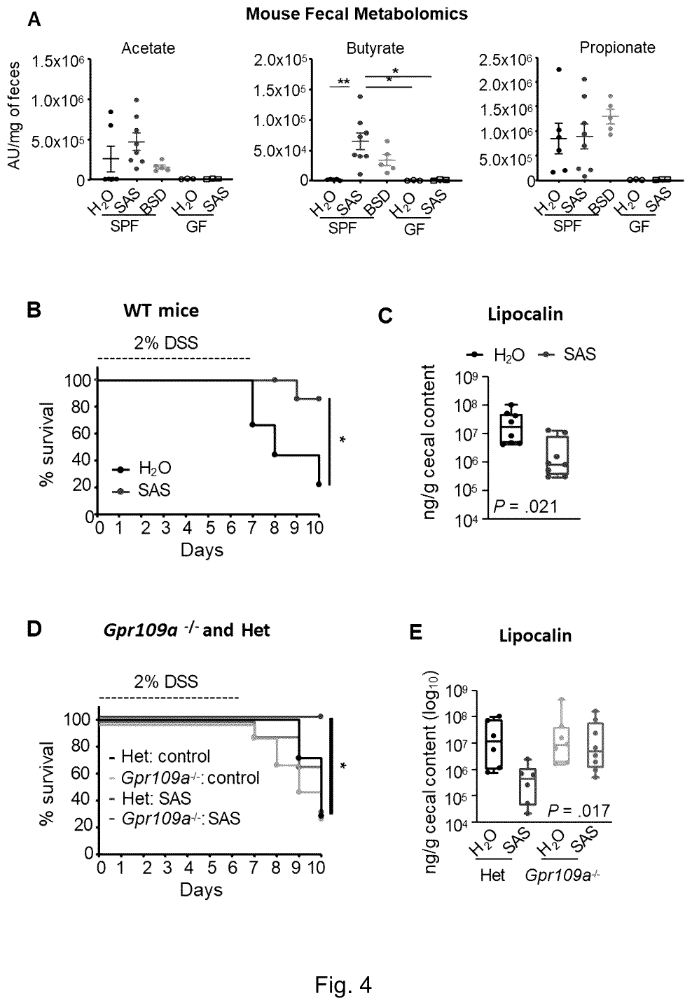

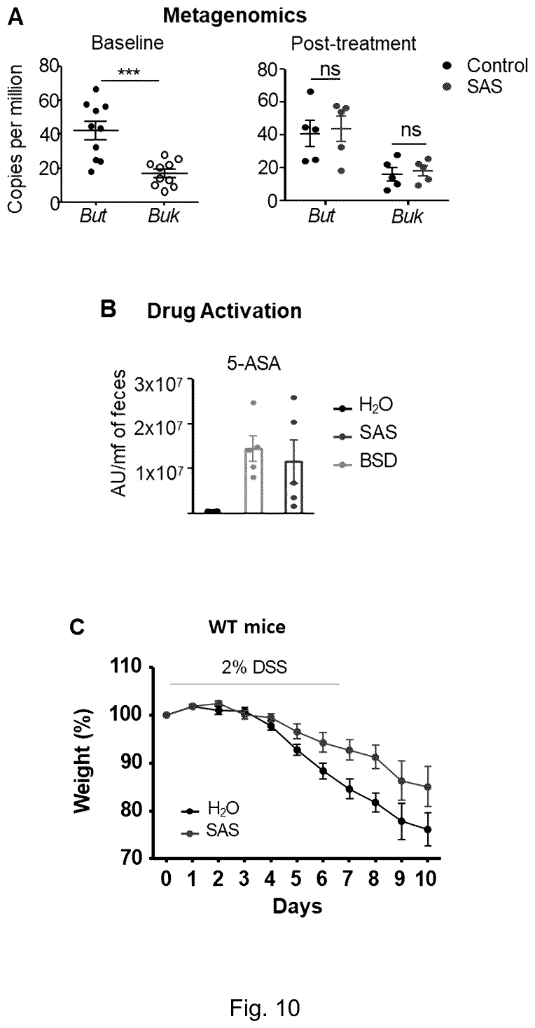

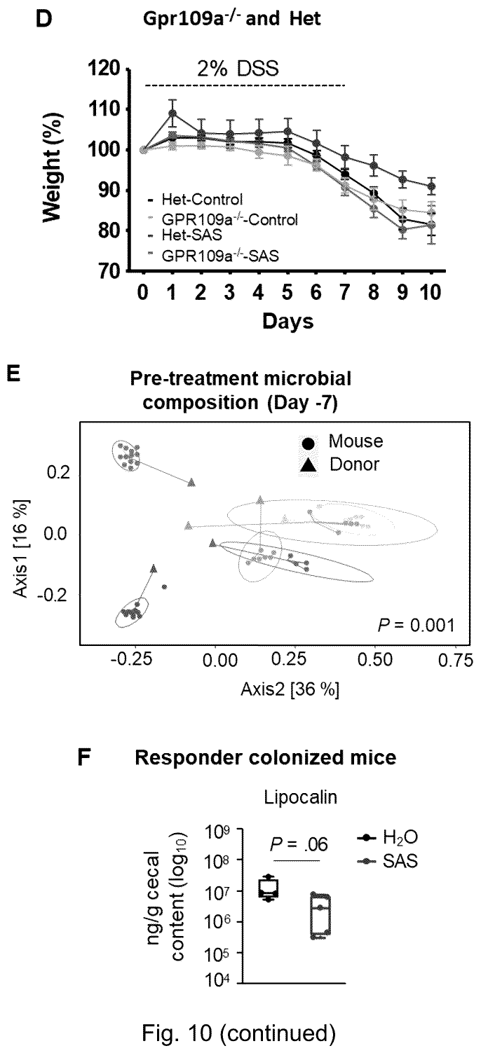

To determine the functional impact on butyrate synthesis in vivo, we treated WT mice with SAS. WT mice received from The Jackson Laboratory were confirmed to have but gene containing microbiota (FIG. 10A). Although no significant change in acetate or propionate was noted upon SAS treatment, SAS treated mice had a significant increase in fecal butyrate levels compared to that observed in H₂O vehicle controls (FIG. 4A). This effect was dependent on the microbiota as GF mice revealed no changes in fecal butyrate levels post-treatment. Further, to test the role for SP in mediating this effect of SAS, SPF mice were also treated with a related 5-ASA without SP called balsalazide (BSD). Both SAS and BSD prodrugs were efficiently converted by the mouse gut microbiome, as demonstrated by equal 5-ASA detection in fecal samples (FIG. 10B). Despite a numerically higher amount of fecal butyrate following BSD, only SAS treated mice had significantly higher fecal butyrate levels compared to controls.

Given the impact of SAS on microbiota-dependent butyrate production in vivo, we next tested the impact of SAS induction of butyrate in an acute model of chemical-induced colitis using dextran sodium sulfate (DSS). SAS treatment significantly reduced mortality (FIG. 4B), attenuated weight loss (FIG. 10C), and reduced cecal lipocalin (FIG. 4C) compared to control treated mice following DSS-induced colitis. To assess if this effect was dependent on butyrate production, mice deficient for the butyrate receptor Gpr109a and heterozygous controls were used. SAS treatment of mice heterozygous for Gpr109a showed significantly improved survival (FIG. 4D), reduced weight loss (FIG. 10D), and decreased intestinal inflammation, as measure by cecal lipocalin (FIG. 4E), compared to control mice not treated with SAS following exposure to DSS. The effect of SAS was dependent on the butyrate receptor, as mice deficient for Gpr109a showed no effect of SAS on survival (FIG. 4D), weight loss (FIG. 10D) or inflammation measured by lipocalin (FIG. 4E).

### Example 6

F. prausnitzii Enhances SAS Protection From Colitis in non-Responder Colonized Mice

To determine if the IBD-pSpA responder and non-responder microbiomes were sufficient to determine differential response to SAS in vivo, gnotobiotic mice were colonized with 6 individual donor microbiomes three weeks prior to metabolomic analysis (FIG. 5A). Consistent with a donor-dependent approach outlined previously⁴⁰, three responder and three non-responder donors were assessed independently with 2-5/mice per donor group. Efficient engraftment was determined by beta diversity analysis (FIG. 10E). Mice colonized with responder microbiomes showed higher abundance of fecal butyrate following SAS treatment (FIG. 5B), while the abundance of butyrate in mice colonized with non-responder microbiome was not changed by treatment with SAS (FIG. 5C).

To assess the microbiome impact on the SAS response in a colitis models, gnotobiotic mice colonized with the 6 individual donor microbiomes were exposed to DSS. Gnotobiotic mice colonized with responder microbiomes from three independent experiments developed weight loss and intestinal inflammation measured by lipocalin, which was reduced by SAS treatment (FIG. 5D, FIG. 10F). In contrast, gnotobiotic mice colonized with non-responder microbiomes from three independent experiments developed weight loss, which was not responsive to SAS treatment (FIG. 5E). To test if F. prausnitzii could enhance the response of non-responder microbiome colonized mice to SAS, we exposed non-responder microbiome colonized mice to F. prausnitzii by oral gavage prior to DSS-colitis induction. Gnotobiotic mice colonized with non-responder microbiomes and exposed to F. prausnitzii were responsive to SAS therapy, as demonstrated by reduced weight loss and cecal lipocalin (FIGS. 5E, F). Although F. prausnitzii alone reduced the weight loss in non-responder microbiome colonized mice, F. prausnitzii+SAS limited weight loss more dramatically and reduced inflammation measured by lipocalin.

### Example 7

### Experimental Model and Study Participant Details

### Human Samples

Sequential subjects with clinical diagnoses of both IBD and ASAS-defined pSpA and an indication for treatment with SAS were prospectively evaluated and longitudinally followed at the Jill Roberts Center for Inflammatory Bowel Disease at Weill Cornell Medicine (WCM, Supplementary Table 1) between December 2015 and March 2020. Eligible patients of any gender were between the ages of 18-80 with ulcerative colitis (UC) or Crohn's disease (CD) and ASAS criteria of pSpA defined as the presence of peripheral arthritis, enthesitis or dactylitis⁵⁰. Patients with active SpA, defined as ASDAS>1.3 and/or BASDAI≥4, were eligible to participate^{51, 52}. Health status exclusion criteria included antibiotic use within 8 weeks prior enrollment, SAS use within 8 months prior enrollment, current pregnancy or lactation, an existing rheumatic disease to which the inflammatory-type joint symptoms were attributed (i.e. systemic lupus erythematosus, rheumatoid arthritis), or an ostomy. Participants who declined therapy or were intolerant of SAS were followed as standard of care controls. SAS subjects were prescribed 2 g increasing to 4 g of SAS daily as tolerated, and 1 mg/day of folic acid. All patients provided voluntary informed consent and the study ethics was approved by the Weill Cornell institutional review board (IRB 1504016115).

A validation cohort of IBD SpA patients was selected from an ongoing biobank study of adult patients with IBD (IRB 1806019340). Exclusion criteria included antibiotic use, an existing rheumatic disease to which the inflammatory-type joint symptoms were attributed (i.e. systemic lupus erythematosus, rheumatoid arthritis), or an ostomy. From a total 442 subjects, we identified 16 subjects with IBD and SpA with sulfasalazine listed as a current medication. 5 subjects had active SpA symptoms and were characterized as non-responders, while 11 subjects had inactive SpA symptoms and characterized as responders. Fecal samples were analyzed by 16S rRNA analysis as described below.

### Mouse Models

Gnotobiotic C57BL/6 wild-type (WT) mice were bred and maintained at WCM Gnotobiotic Mouse facility. Specific pathogen free (SPF) C57BL/6 WT mice were ordered from Jackson Laboratory. C57BL/6 Gpr109a^{-/-} mice were obtained from Marcel van den Brink laboratory and were bred and maintained under SPF conditions at WCM.

For induction of chemical colitis, 2% dextran sulfate sodium (DSS) (wt/vol) (M.W. 40,000-50,000; Affymetrix, Santa Clara, CA) was added to drinking water and administered ad libitum for 7 days. Mice were then monitored daily for weight loss and survival. Mouse Lipocalin-2 was measured in the cecal content supernatant using sandwich enzyme-linked immunosorbent assay (R&D Systems, Minneapolis, MN). Mice were treated with 300 mg/kg/day of SAS (Sigma-Aldrich, St. Louis, MO), balsalazide (BSD; TCI, America) or water vehicle control, as previously described²⁵.

For gnotobiotic experiments, germ-free (GF) mice were gavaged with 1 g of homogenized patient fecal samples diluted in 10 ml of reduced PBS under anaerobic conditions, as previously described⁵³. Mice gavaged with ATCC 27768 F. prausnitzii received 1×10⁹ colony forming units (CFU) grown under anaerobic conditions at log-phase in Yeast Casitone Fatty Acids Broth with Carbohydrate media (YCFAC, Anaerobe Systems, Morgan Hill, CA).

All animal experiments were performed with 6-8-week-old WT, Gpr109a^{-/-} and heterozygous littermate mice. Both female and male mice were used with random and equal assignment of same sex to each experimental group. All animal studies were carried out in accordance with protocols approved by the Institutional Animal Care and Use Committee at WCM.

### Method Details

### Endpoint Analysis

The primary endpoint of our analysis was clinical response in BASDAI, defined conservatively as a reduction in BASDAI of >2. Secondary endpoints were clinical response in ASDAS and Harvey Bradshaw Index (HBI) for CD defined as reduction in ASDAS of ≥1.1 at week 12 post-study initiation⁵¹ or a decrease in HBI of ≥3⁵⁴, respectively. Exploratory analysis included absolute reduction in BASDAI, ASDAS, CRP, and HBI.

### Fecal DNA Extraction

Upon collection, stool samples were promptly divided into 2 mL aliquots and stored at −80° C. For microbiome analysis, DNA was isolated from approximately 250 mg of stool samples using the DNeasy PowerLyzer PowerSoil Kit (Qiagen, Hilden, Germany), following manufacturer's instructions.

### Metagenomic Analysis

To evaluate the impact of SAS on the fecal microbiome composition and its functional potential, we performed metagenomic sequencing. Metagenomic library was constructed with Illumina barcodes from the Nextera XT kit (Illumina, San Diego, CA) and then loaded into Illumina Hiseq 4000 platform using 2×150 nucleotide pair-ending sequencing protocol at Weill Cornell Microbiome Core. Sequencing data analysis is detailed in supplemental methods. Raw data was processed by KneadData (bitbucket.org/biobakery/kneaddata) to remove human contaminant reads. To filter out low quality sequence reads, sequences shorter than 50 bp and Illumina adapters reads, Trimmomatic 55 was used. Samples from three patients failed metagenomic library processing (BASDAI responders n=2, BASDAI non-responders n=1). After quality control, samples averaged 4 million reads (mean=4,286,719.1, standard error of the mean-SEM±251, 131.1). Taxonomic profiling was determined by MetaPhlAn2 pipeline 56. Microbial abundances were calculated with MetaPhlAn2, following Bowtie2 57 alignment to the MetaPhlAn2 database. Microbial functional potential profiling was determined by HUMAnN258. For gene profiling, DIAMOND 59 was used to map metagenome reads against UniRef90 60. Hits were counted for each gene family and normalized for length and alignment quality. For pathway profiling, gene family abundances were combined into structured pathways from MetaCyc 61 and then sum-normalized to copies per million (CPM).

Metabolomic analysis

To determine drug fecal levels and its impact on fecal metabolites, fecal samples were treated with cold 80% methanol for 4 hours at 4° C. Samples were then centrifuged at 14,000 g for 20 min at 4° C. Supernatants were transferred to a sterile 1.5 ml tube and stored at-80° C. Bacterial supernatants were treated with 4X volume of the 80% methanol. Polar metabolomic analysis was performed by the WCM Proteomics and Metabolomics Core for target polar metabolomics analysis by LC-MS. Metabolites were identified using an in-house library established using chemical standards. Identification required exact mass (within 5ppm) and standard retention times. To determine short chain fatty acids (SCFAs) fecal levels, target metabolomics were performed using liquid chromatography-quadruple-time of flight mass spectrometry as previously described⁶². Relative metabolite quantitation was performed based on peak area for each metabolite evaluated and normalized by sample weight.

16S rRNA Sequencing

To evaluate if the fecal microbiome of patients were efficiently transplanted into germ-free mice, both patient-donors and mice fecal samples were subjected to 16S rRNA sequencing at WCM Microbiome Core Facility. Briefly, after DNA isolation, the 16S V4 and V5regions were amplified as previously described (earthmicrobiome.org). Amplicons were then sequenced in an Illumina MiSeq platform using the 2×250 bp paired-end protocol. Read pairs were processed using DADA2⁶³ with forward truncation length of 240, reverse truncation length of 160, and otherwise default parameters, VSEARCH⁶⁴, and the SILVA Database⁶⁵ (version 138) for taxonomic assignment. This produced a rarefied amplicon sequence variant (ASV) tables and phylogenetic tree (sequence depth of 12000 reads for human data and 10146 reads for mouse data) for downstream microbiome analysis. 16S rRNA seq analyses were performed in R studio (Boston, MA). Beta diversity was calculated using R package ‘phyloseq’⁶⁶ based on unweighted uniFrac, while plots were constructed in ‘ggplot2’ or GraphPad (San Diego, CA).

### In Vitro Assays and Bacterial RNA-seq

F. prausnitzii ATCC 27768 or E. rectale DSM 17629 was grown at 37° C., anaerobically in YCFAC media (for transcriptomics) or RCM media (for metabolomics) with sodium hydroxide (NaOH) vehicle control (0.5M; Sigma-Aldrich, St. Louis, MO), sulfapyridine (500, 1000, 2000 μM; Sigma-Aldrich, St. Louis, MO) and/or folate (566 μM; Sigma-Aldrich, St. Louis, MO), adapted from Nayak et al., 2021³⁸. CFU from F. prausnitzii cultures were assessed anaerobically in YCFA agar. Growth was measured by absorbance, OD₆₀₀, on SpectraMax Plus (Molecular Devices, San Jose, CA). RNA isolation was performed using RNeasy PowerMicrobiome kit (Qiagen, Hilden, Germany) following the manufacture instructions. RNA was then quantified by Nanodrop prior to reverse transcription with qScript cDNA SuperMix kit (Quantabio, Beverly, MA) or RNAseq library preparation.

RNA-seq library preparation and sequencing were performed by WCM Genomics Core. For library preparation, the Illumina Stranded Total RNA Prep kit was used. High-throughput sequencing was performed using the PE 2×50 cycles protocol in the NovaSeq 6000 Illumina platform targeting 10 million reads/sample. Bioinformatics and statistical analysis were then performed by the WCM Bioinformatics Core. Samples were aligned with STAR⁶⁷ to NCBI reference genome GCF_000154385.1 (F. prausnitzii M21/2). Quality control was performed with QoRTs⁶⁸ on aligned BAM files. Counts per gene were then calculated using featureCounts⁶⁹. BioCyc⁷⁰ analyses were performed with clusterProfiler to identify pathways/gene sets that are overrepresented among the differentially expressed genes.

### Quantitative RT-PCR

qPCR was performed on an Applied BioSciences Quant Studio 6 Flex Real-time PCR (Applied Biosystems, Waltham, MA) using PerfeCTa SYBR Green Fast mix, Low ROX (Quantabio, Beverly, MA). The following primers were used: for 16S rRNA gene −515F 5′ GTGYCAGCMGCCGCGGTAA 3′ (SEQ ID NO:1), −926R 5′ CCGYCAATTYMTTTRAGTTT 3′ (SEQ ID NO:2)⁷¹ F. prausnitzii but gene F: 5′ GTGGATGCCTTTGTGGATATTG 3′ SEQ ID NO:3), R: 5′ CCGGGTTATCGTTCAGGTAATC 3′ (SEQ ID NO:4) and E. rectale but gene F: 5′ CTCAGCAGATGAGGCAGTAAAG 3′, SEQ ID NO:5) R: 5′ GGAAGCTCTGAGTAACGGATTG 3′ (SEQ ID NO:6) (designed on the PrimerQuest Tool from IDT, Coralville, IA). The thermocycler program was as follows: initial cycle of 95° C. for 60 s, followed by 40 PCR cycles at 95° C. for 5 s, 60° C. for 15 s, 72° C. for 15 s. Relative levels of the but target gene was determined by calculating the Δ Ct to the conserved 16S rRNA gene expression.

### Quantification and Statistical Analysis

For endpoint analysis, Fisher's exact test implemented in JMP 14 (SAS Institute Inc., Cary, NC) was used when proportions were compared between binary variables. To determine significance of categorical variables, Student's t test was applied if two groups were compared, otherwise two-way ANOVA test was used. For non-parametric data, Wilcoxon matched-pairs signed-rank test was calculated when paired analyses were carried, otherwise Mann-Whitney test was applied. If required, Bonferroni or Tukey tests were applied for multiple comparison correction. Metagenomic and transcriptomic analyses were performed in R studio (Boston, MA) using R package ‘phyloseq’⁶⁶ and plots were constructed in ‘ggplot2’ or GraphPad (San Diego, CA). Differential gene expression analysis was performed with DESeq2⁷². Differential taxa or pathway abundance was assessed using linear discriminant analysis (LDA) effect size (LEfSe)³¹. Correlation between two continuous variables (e.g. fecal drug levels, Δ BASDAI, alpha diversity) was determined with linear regression models. For discriminating responders from non-responders, we fit linear regression models on the relative abundances of select taxa and constructed corresponding ROC curves. Area under the curve (AUC) and P values were computed using R.

### Discussion of Examples

Although SpA is the most common extra-intestinal manifestation in IBD,

previous limitations in both diagnostic and therapeutic strategies for IBD-pSpA frequently delay treatment and response rates. This disclosure demonstrates that SAS therapy leads to improvement in peripheral SpA symptoms of IBD patients as measured by BASDAI and ASDAS. Previous studies identified differences in the fecal microbiome of subjects with IBD-SpA compared to SpA alone including the expansion of Adherent-Invasive E. coli¹² and R. gnavus¹¹. In contrast, this disclosure provides evidence that the baseline microbiome can discriminate responders and non-responders to SAS therapy. F. prausnitzii is the most robust taxa discriminating SAS responders from non-responders in our validation cohort. F. prausnitzii is notably reduced in SpA patients with a history of IBD¹⁴ and may provide a key link between disease biology and SAS response.

The microbiome has long been considered a target of SAS therapy, but the specific taxa and mechanisms involved in drug efficacy have not been defined. Previous studies have suggested that SAS acts as a direct antimicrobial on gut bacteria^{24, 25}. Recent studies with methotrexate also revealed an antifolate/antimicrobial effect³⁸. Furthermore, a recent report revealed that microbiome metabolism of 5-aminosalicylate stratifies mesalamine responders from non-responders⁴¹. In this disclosure, metagenomic analysis revealed distinct baseline gene markers that correlate with SAS response including sub-pathways related to the basic cellular processes of carbohydrate degradation and SCFA synthesis characteristic of a healthy microbiome³². Moreover, the enrichment of the butyrate producers F. prausnitzii, A. onderdonkii and R. inulinivorans in SAS-responders may identify a cohort with higher anti-inflammatory potential for the induction of IL-10 or regulatory T cells⁴²⁻⁴⁴. Adding to these associations with baseline composition, this disclosure reveals a mechanism by which SAS acts as a xenobiotic on F. prausnitzii to modulate transcription of genes associated with butyrate production and boost production of butyrate in vivo. Enhanced production of butyrate is required for SAS-induced, microbe-dependent protection from experimental colitis in mice. Furthermore, SAS-enhanced production of butyrate was also seen in gnotobiotic mice colonized with responder microbiome, and not non-responder microbiomes, suggesting the sufficiency of the microbiome to imprint this response. Additional colonization of non-responder microbiome colonized mice with lead responder-marker taxa F. prausnitzii was sufficient to recover SAS protection against experimental colitis. The results of this disclosure demonstrate the ability of SAS to promote the production of SCFA in an SP-dependent fashion and to promote protection against experimental colitis, which is considered extendable to human disease. Previous reports have defined a role for gut-derived butyrate in reducing inflammatory arthritis^{46, 47}, but the cellular effectors mediating systemic inflammation in IBD-associated pSpA still need to be defined. Collectively, the results presented in this disclosure reveal a functional interaction of SAS with specific bacterial taxa that allow not only stratification of patients likely to respond to SAS therapy, but also guide interventional microbial therapies to enhance the efficacy of SAS for IBD-SpA, as described herein.

Folate plays an important role in one carbon metabolism in multiple cellular reactions that are required to produce amino acids, thymidine and purines^{48, 49}. The inhibition of bacterial de novo folate biosynthesis by SP moiety of SAS can therefore activate folate stress pathways^{26,37}. F. prausnitzii encodes a folP-like enzyme called MBL-fold metallohydrolase which is likely a target of SP³⁶. Consistent with cellular sensing of foll inhibition, the disclosure reveals upregulation in folC and folT following SP treatment of F. prausnitzii. Similarly supporting the role for folate stress pathways, SP induction of butyrate synthesis genes was suppressed by excess folate. Other commensals, such as E. rectale, which do produce butyrate and encode but, do not regulate butyrate synthesis in response to SP. E. rectale does not encode folP, which may confer specificity of the SP effect on butyrate synthesis. Even though all patients in this study received folate supplementation, oral supplementation with folate is primarily absorbed in the small intestine while SAS prodrug cleavage by microbial azoreductases to release SP and 5-ASA occurs distally in the colon³⁹. Consistent with a functional antifolate effect of SP, evidence of a folate trap in the fecal metabolome of responders (but not non-responders) support the role for the microbiome in the clinical efficacy of SAS for pSpA. The results presented in this disclosure support the use of the described microbial biomarkers in stratifying therapeutic responses to SAS, as well as the described microbial-based therapies in optimizing and enhancing medication efficacy.

References-this reference listing is not an indication that any particular reference is material to patentability.
1. Ossum A M, Palm O, Lunder A K, Cvancarova M, Banitalebi H, Negard A, Hoie O, Henriksen M, Moum B A, Hoivik M L, Group IS. Ankylosing Spondylitis and Axial Spondyloarthritis in Patients With Long-term Inflammatory Bowel Disease: Results From 20

Years of Follow-up in the IBSEN Study. J Crohns Colitis. 2018;12(1):96-104. doi: 10.1093/ecco-jcc/jjx126. PubMed PMID: 28961700.
2. van Erp S J, Brakenhoff L K, van Gaalen F A, van den Berg R, Fidder H H, Verspaget H W, Huizinga T W, Veenendaal R A, Wolterbeek R, van der Heijde D, van der Meulen-de Jong A E, Hommes D W. Classifying Back Pain and Peripheral Joint Complaints in Inflammatory Bowel Disease Patients: A Prospective Longitudinal Follow-up Study. J Crohns Colitis. 2016;10(2):166-75. doi: 10.1093/ecco-jcc/jjv195. PubMed PMID: 26512134.
3. Palm O, Moum B, Jahnsen J, Gran J T. The prevalence and incidence of peripheral arthritis in patients with inflammatory bowel disease, a prospective population-based study (the IBSEN study). Rheumatology (Oxford). 2001;40(11):1256-61. doi: 10.1093/rheumatology/40.11.1256. PubMed PMID: 11709609.
4. Orchard T R, Wordsworth B P, Jewell D P. Peripheral arthropathies in inflammatory bowel disease: their articular distribution and natural history. Gut. 1998;42(3):387-91. doi: 10.1136/gut.42.3.387. PubMed PMID: 9577346; PMCID: PMC1727027.
5. Ditisheim S, Fournier N, Juillerat P, Pittet V, Michetti P, Gabay C, Finckh A, Swiss IBDCSG. Inflammatory Articular Disease in Patients with Inflammatory Bowel Disease: Result of the Swiss IBD Cohort Study. Inflammatory bowel diseases. 2015;21(11):2598-604. doi: 10.1097/MIB.0000000000000548. PubMed PMID: 26244648.
6. Karreman M C, Luime J J, Hazes J M W, Weel A. The Prevalence and Incidence of Axial and Peripheral Spondyloarthritis in Inflammatory Bowel Disease: A Systematic Review and Meta-analysis. J Crohns Colitis. 2017; 11(5):631-42. doi: 10.1093/ecco-jcc/jjw199. PubMed PMID: 28453761.
7. Harbord M, Annese V, Vavricka S R, Allez M, Barreiro-de Acosta M, Boberg K M, Burisch J, De Vos M, De Vries A M, Dick A D, Juillerat P, Karlsen T H, Koutroubakis I, Lakatos P L, Orchard T, Papay P, Raine T, Reinshagen M, Thaci D, Tilg H, Carbonnel F, European Cs, Colitis O. The First European Evidence-based Consensus on Extra-intestinal Manifestations in Inflammatory Bowel Disease. J Crohns Colitis. 2016;10(3):239-54. doi: 10.1093/ecco-jcc/jjv213. PubMed PMID: 26614685; PMCID: PMC4957476.
8. Kumar A, Lukin D, Battat R, Schwartzman M, Mandl L A, Scherl E, Longman R S. Defining the phenotype, pathogenesis and treatment of Crohn's disease associated spondyloarthritis. J Gastroenterol. 2020;55(7):667-78. Epub 20200504. doi: 10.1007/s00535-020-01692-w. PubMed PMID: 32367294; PMCID: PMC7297835.
9. Tito R Y, Cypers H, Joossens M, Varkas G, Van Praet L, Glorieus E, Van den Bosch F, De Vos M, Raes J, Elewaut D. Brief Report: Dialister as a Microbial Marker of Disease Activity in Spondyloarthritis. Arthritis Rheumatol. 2017;69(1):114-21. Epub 20161201. doi: 10.1002/art.39802. PubMed PMID: 27390077.
10. Muniz Pedrogo D A, Chen J, Hillmann B, Jeraldo P, Al-Ghalith G, Taneja V, Davis J M, Knights D, Nelson H, Faubion W A, Raffals L, Kashyap P C. An Increased Abundance of Clostridiaceae Characterizes Arthritis in Inflammatory Bowel Disease and Rheumatoid Arthritis: A Cross-sectional Study. Inflamm Bowel Dis. 2019;25(5):902-13. doi: 10.1093/ibd/izy318. PubMed PMID: 30321331; PMCID: PMC6458525.
11. Breban M, Tap J, Leboime A, Said-Nahal R, Langella P, Chiocchia G, Furet J P, Sokol H. Faecal microbiota study reveals specific dysbiosis in spondyloarthritis. Ann Rheum Dis. 2017;76(9):1614-22. Epub 2017 Jun. 14. doi: 10.1136/annrheumdis-2016-211064. PubMed PMID: 28606969.
12. Viladomiu M, Kivolowitz C, Abdulhamid A, Dogan B, Victorio D, Castellanos J G, Woo V, Teng F, Tran N L, Sczesnak A, Chai C, Kim M, Diehl G E, Ajami N J, Petrosino J F, Zhou X K, Schwartzman S, Mandl L A, Abramowitz M, Jacob V, Bosworth B, Steinlauf A, Scherl E J, Wu H J, Simpson K W, Longman R S. IgA-coated E. coli enriched in Crohn's disease spondyloarthritis promote TH17-dependent inflammation. Sci Transl Med. 2017;9(376). Epub 2017 Feb. 10. doi: 10.1126/scitranslmed.aaf9655. PubMed PMID: 28179509.
13. Viladomiu M, Metz M L, Lima S F, Jin W B, Chou L, Bank JRILC, Guo C J, Diehl G E, Simpson K W, Scherl E J, Longman R S. Adherent-invasive E. coli metabolism of propanediol in Crohn's disease regulates phagocytes to drive intestinal inflammation. Cell Host Microbe. 2021;29(4):607-19 e8. Epub 20210203. doi: 10.1016/j.chom.2021.01.002. PubMed PMID: 33539767; PMCID: PMC8049981.
14. Berland M, Meslier V, Berreira Ibraim S, Le Chatelier E, Pons N, Maziers N, Thirion F, Gauthier F, Plaza Onate F, Furet J P, Leboime A, Said-Nahal R, Levenez F, Galleron N, Quinquis B, Langella P, Ehrlich S D, Breban M. Both Disease Activity and HLA-B27 Status Are Associated With Gut Microbiome Dysbiosis in Spondyloarthritis Patients. Arthritis Rheumatol. 2023;75(1):41-52. Epub 20221119. doi: 10.1002/art.42289. PubMed PMID: 35818337; PMCID: PMC10099252.
15. J. J. Misiewicz M. B. BSLJEL-JBC, M. R. C. P. A. M. Connell M. B. Glasg., M. R. C. P. E. (MEMBERS OF SCIENTIFIC STAFF, MEDICAL RESEARCH COUNCIL GASTROENTEROLOGY RESEARCH UNIT, CENTRAL MIDDLESEX HOSPITAL, LONDON)1J. H. Baron D. M. Oxon., M.R.C.P. (SENIOR REGISTRAR, MIDDLESEX HOSPITAL, LONDON) F. Avery Jones M.D. Lond., F.R.C.P. (CONSULTANT GASTROENTEROLOGIST). CONTROLLED TRIAL OF SULPHASALAZINE IN MAINTENANCE THERAPY FOR ULCERATIVE COLITIS. The Lancet. 1965;285(7378):185-8. doi: doi.org/10.1016/S0140-6736 (65) 90972-4.
16. Baron J H, Connell A M, Lennard-Jones J E, Jones F A. Sulphasalazine and salicylazosulphadimidine in ulcerative colitis. Lancet. 1962;1(7239):1094-6. doi: 10.1016/s0140-6736(62)92080-9. PubMed PMID: 13865153.
17. Dick A P, Grayson M J, Carpenter R G, Petrie A. Controlled Trial of Sulphasalazine in the Treatment of Ulcerative Colitis. Gut. 1964;5(5):437-42. doi: 10.1136/gut.5.5.437. PubMed PMID: 14218553; PMCID: PMC1552152.
18. Dissanayake A S, Truelove S C. Proceedings: A controlled therapeutic trial of long-term maintenance treatment of ulcerative colitis with sulphasalazine (salazopyrin). Gut. 1973;14(10):818. PubMed PMID: 4148469.
19. Svartz M. The treatment of 124 cases of ulcerative colitis with salazopyrine and attempts of desensibilization in cases of hypersensitiveness to sulfa. Acta Med Scand. 1948;131 (Suppl 206):465-72. doi: 10.1111/j.0954-6820.1948.tb12083.x. PubMed PMID: 18881171.
20. Peppercorn M A, Goldman P. The role of intestinal bacteria in the metabolism of salicylazosulfapyridine. J Pharmacol Exp Ther. 1972;181(3):555-62. PubMed PMID: 4402374.
21. Byndloss M X, Olsan E E, Rivera-Chavez F, Tiffany C R, Cevallos S A, Lokken K L, Torres T P, Byndloss A J, Faber F, Gao Y, Litvak Y, Lopez C A, Xu G, Napoli E, Giulivi C, Tsolis R M, Revzin A, Lebrilla C B, Baumler A J. Microbiota-activated PPAR-gamma signaling inhibits dysbiotic Enterobacteriaceae expansion. Science. 2017;357(6351):570-5. doi: 10.1126/science.aam9949. PubMed PMID: 28798125; PMCID: PMC5642957.
22. Azad Khan A K, Piris J, Truelove S C. An experiment to determine the active therapeutic moiety of sulphasalazine. Lancet. 1977;2(8044):892-5. doi: 10.1016/s0140-6736 (77) 90831-5. PubMed PMID: 72239.
23. Clegg D O, Reda D J, Abdellatif M. Comparison of sulfasalazine and placebo for the treatment of axial and peripheral articular manifestations of the seronegative spondylarthropathies: a Department of Veterans Affairs cooperative study. Arthritis and rheumatism. 1999;42(11): 2325-9. Epub 1999 Nov. 11. doi: 10.1002/1529-0131 (199911)42:11<2325::AID-ANR10>3.0.CO;2-C. PubMed PMID: 10555027.
24. Kanerud L, Scheynius A, Nord C E, Hafström I. Effect of sulphasalazine on gastrointestinal microflora and on mucosal heat shock protein expression in patients with rheumatoid arthritis. Br J Rheumatol. 1994;33(11):1039-48. doi: 10.1093/rheumatology/33.11.1039. PubMed PMID: 7981991.
25. Zheng H, Chen M, Li Y, Wang Y, Wei L, Liao Z, Wang M, Ma F, Liao Q, Xie Z. Modulation of Gut Microbiome Composition and Function in Experimental Colitis Treated with Sulfasalazine. Front Microbiol. 2017;8:1703. Epub 2017 Sep. 7. doi: 10.3389/fmicb.2017.01703. PubMed PMID: 28936203; PMCID: PMC5594074.
26. Park H B, Wei Z, Oh J, Xu H, Kim C S, Wang R, Wyche T P, Piizzi G, Flavell R A, Crawford J M. Sulfamethoxazole drug stress upregulates antioxidant immunomodulatory metabolites in Escherichia coli. Nat Microbiol. 2020;5(11):1319-29. Epub 2020 Jul. 29. doi: 10.1038/s41564-020-0763-4. PubMed PMID: 32719505; PMCID: PMC7581551.
27. van der Heijde D, Sieper J, Maksymowych W P, Dougados M, Burgos-Vargas R, Landewe R, Rudwaleit M, Braun J, Assessment of SpondyloArthritis international S. 2010 Update of the international ASAS recommendations for the use of anti-TNF agents in patients with axial spondyloarthritis. Ann Rheum Dis. 2011;70(6):905-8. doi: 10.1136/ard.2011.151563.PubMed PMID: 21540200.
28. Garrett S, Jenkinson T, Kennedy L G, Whitelock H, Gaisford P, Calin A. A new approach to defining disease status in ankylosing spondylitis: the Bath Ankylosing Spondylitis Disease Activity Index. J Rheumatol. 1994;21(12):2286-91. PubMed PMID: 7699630.
29. Lai D, Funez-Depagnier G, Duenas-Bianchi L, Lavergne A, Battat R, Ahmed W, Schwartzman M, Lima S, Khan S, Chong P S, Sonnenberg G, Artis D, Lukin D, Scherl E, Longman R S. Joint Disease Activity in Inflammatory Bowel Disease-associated Peripheral Spondyloarthritis Stratifies Therapeutic Response. Gastro Hep Adv. 2022;1(2):137-40. Epub 20220207. doi: 10.1016/j.gastha.2021.12.002. PubMed PMID: 35441160; PMCID: PMC9015680.
30. Lukas C, Landewe R, Sieper J, Dougados M, Davis J, Braun J, van der Linden S, van der Heijde D, Assessment of SpondyloArthritis international S. Development of an ASAS-endorsed disease activity score (ASDAS) in patients with ankylosing spondylitis. Ann Rheum Dis. 2009;68(1):18-24. doi: 10.1136/ard.2008.094870. PubMed PMID: 18625618.
31. Segata N, Izard J, Waldron L, Gevers D, Miropolsky L, Garrett W S, Huttenhower C. Metagenomic biomarker discovery and explanation. Genome Biol. 2011; 12(6):R60. Epub 2011 Jun. 24. doi: 10.1186/gb-2011-12-6-r60. PubMed PMID: 21702898; PMCID: PMC3218848.
32. Morgan X C, Tickle T L, Sokol H, Gevers D, Devaney K L, Ward D V, Reyes J A, Shah S A, LeLeiko N, Snapper S B, Bousvaros A, Korzenik J, Sands B E, Xavier R J, Huttenhower C. Dysfunction of the intestinal microbiome in inflammatory bowel disease and treatment. Genome Biol. 2012;13(9):R79. doi: 10.1186/gb-2012-13-9-r79. PubMed PMID: 23013615; PMCID: PMC3506950.
33. Rao K S, Albro M, Dwyer T M, Frerman F E. Kinetic mechanism of glutaryl-CoA dehydrogenase. Biochemistry. 2006;45(51):15853-61. Epub 20061202. doi: 10.1021/bi0609016. PubMed PMID: 17176108.
34. Erb T J, Brecht V, Fuchs G, Muller M, Alber B E. Carboxylation mechanism and stereochemistry of crotonyl-CoA carboxylase/reductase, a carboxylating enoyl-thioester reductase. Proc Natl Acad Sci U S A. 2009;106(22):8871-6. Epub 20090520. doi: 10.1073/pnas.0903939106. PubMed PMID: 19458256; PMCID: PMC2689996.
35. Klotz U. Clinical pharmacokinetics of sulphasalazine, its metabolites and other prodrugs of 5-aminosalicylic acid. Clin Pharmacokinet. 1985;10(4):285-302. doi: 10.2165/00003088-198510040-00001. PubMed PMID: 2864155.
36. Xu H, Aurora R, Rose G D, White R H. Identifying two ancient enzymes in Archaea using predicted secondary structure alignment. Nat Struct Biol. 1999;6(8):750-4. doi: 10.1038/11525. PubMed PMID: 10426953.
37. Guzzo M B, Nguyen H T, Pham T H, Wyszczelska-Rokiel M, Jakubowski H, Wolff K A, Ogwang S, Timpona J L, Gogula S, Jacobs M R, Ruetz M, Kräutler B, Jacobsen D W, Zhang G F, Nguyen L. Methylfolate Trap Promotes Bacterial Thymineless Death by Sulfa Drugs. PLoS Pathog. 2016;12(10):e1005949. Epub 2016 Oct. 19. doi: 10.1371/journal.ppat. 1005949. PubMed PMID: 27760199; PMCID: PMC5070874.
38. Nayak R R, Alexander M, Deshpande I, Stapleton-Gray K, Rimal B, Patterson A D, Ubeda C, Scher J U, Turnbaugh P J. Methotrexate impacts conserved pathways in diverse human gut bacteria leading to decreased host immune activation. Cell Host Microbe. 2021;29(3):362-77.e11. Epub 20210112. doi: 10.1016/j.chom.2020.12.008. PubMed PMID: 33440172; PMCID: PMC7954989.
39. svarz n. Salazopyrin, a new sulfanilamide preparation. A. Therapeutic Results in Rheumatic Polyarthritis. B. Therapeutic Results in Ulcerative Colitis. C. Toxic Manifestations in Treatment with Sulfanilamide Preparations. Acta Med Scand. 1942;CX. doi: doi.org/10.1111/j.0954-6820.1942.tb06841.x.
40. Walter J, Armet A M, Finlay B B, Shanahan F. Establishing or Exaggerating Causality for the Gut Microbiome: Lessons from Human Microbiota-Associated Rodents. Cell. 2020; 180(2):221-32. doi: 10.1016/j.cell.2019.12.025. PubMed PMID: 31978342.
41. Mehta R S, Mayers J R, Zhang Y, Bhosle A, Glasser N R, Nguyen L H, Ma W, Bae S, Branck T, Song K, Sebastian L, Pacheco J A, Seo H S, Clish C, Dhe-Paganon S, Ananthakrishnan A N, Franzosa E A, Balskus E P, Chan A T, Huttenhower C. Gut microbial metabolism of 5-ASA diminishes its clinical efficacy in inflammatory bowel disease. Nat Med. 2023;29(3):700-9. Epub 20230223. doi: 10.1038/s41591-023-02217-7. PubMed PMID: 36823301.
42. Sokol H, Pigneur B, Watterlot L, Lakhdari O, Bermudez-Humaran L G, Gratadoux J J, Blugeon S, Bridonneau C, Furet J P, Corthier G, Grangette C, Vasquez N, Pochart P, Trugnan G, Thomas G, Blottiere H M, Dore J, Marteau P, Seksik P, Langella P. Faecalibacterium prausnitzii is an anti-inflammatory commensal bacterium identified by gut microbiota analysis of Crohn disease patients. Proc Natl Acad Sci U S A. 2008; 105(43):16731-6. Epub 2008 Oct. 22. doi: 0804812105 [pii] 10.1073/pnas.0804812105. PubMed PMID: 18936492; PMCID: 2575488.
43. Atarashi K, Tanoue T, Shima T, Imaoka A, Kuwahara T, Momose Y, Cheng G, Yamasaki S, Saito T, Ohba Y, Taniguchi T, Takeda K, Hori S, Ivanov I I, Umesaki Y, Itoh K, Honda K. Induction of colonic regulatory T cells by indigenous Clostridium species. Science. 2011;331(6015):337-41. Epub 2010 Dec. 23. doi: 10.1126/science.1198469. PubMed PMID: 21205640; PMCID: PMC3969237.
44. Furusawa Y, Obata Y, Fukuda S, Endo T A, Nakato G, Takahashi D, Nakanishi Y, Uetake C, Kato K, Kato T, Takahashi M, Fukuda N N, Murakami S, Miyauchi E, Hino S, Atarashi K, Onawa S, Fujimura Y, Lockett T, Clarke J M, Topping D L, Tomita M, Hori S, Ohara O, Morita T, Koseki H, Kikuchi J, Honda K, Hase K, Ohno H. Commensal microbe-derived butyrate induces the differentiation of colonic regulatory T cells. Nature. 2013;504(7480):446-50. Epub 2013 Nov. 15. doi: 10.1038/nature12721. PubMed PMID: 24226770.
45. Quevrain E, Maubert M A, Michon C, Chain F, Marquant R, Tailhades J, Miquel S, Carlier L, Bermudez-Humaran L G, Pigneur B, Lequin O, Kharrat P, Thomas G, Rainteau D, Aubry C, Breyner N, Afonso C, Lavielle S, Grill J P, Chassaing G, Chatel J M, Trugnan G, Xavier R, Langella P, Sokol H, Seksik P. Identification of an anti-inflammatory protein from Faecalibacterium prausnitzii, a commensal bacterium deficient in Crohn's disease. Gut. 2016;65 (3): 415-25. Epub 20150604. doi: 10.1136/gutjnl-2014-307649. PubMed PMID: 26045134; PMCID: PMC5136800.
46. Rosser E C, Piper C J M, Matei D E, Blair P A, Rendeiro A F, Orford M, Alber D G, Krausgruber T, Catalan D, Klein N, Manson J J, Drozdov I, Bock C, Wedderburn L R, Eaton S, Mauri C. Microbiota-Derived Metabolites Suppress Arthritis by Amplifying Aryl-Hydrocarbon Receptor Activation in Regulatory B Cells. Cell Metab. 2020;31(4):837-51.e10. Epub 20200325. doi: 10.1016/j.cmet.2020.03.003. PubMed PMID: 32213346; PMCID: PMC7156916.
47. Hui W, Yu D, Cao Z, Zhao X. Butyrate inhibit collagen-induced arthritis via Treg/IL-10/Th17 axis. Int Immunopharmacol. 2019;68:226-33. Epub 20190116. doi: 10.1016/j.intimp.2019.01.018. PubMed PMID: 30660077.
48. Ducker G S, Rabinowitz J D. One-Carbon Metabolism in Health and Disease. Cell Metab. 2017;25(1):27-42. Epub 2016 Sep. 15. doi: 10.1016/j.cmet.2016.08.009. PubMed PMID: 27641100; PMCID: PMC5353360.
49. Selhub J. Folate, vitamin B12 and vitamin B6 and one carbon metabolism. J Nutr Health Aging. 2002;6(1):39-42. PubMed PMID: 11813080.
50. Rudwaleit M, van der Heijde D, Landewe R, Akkoc N, Brandt J, Chou C T, Dougados M, Huang F, Gu J, Kirazli Y, Van den Bosch F, Olivieri I, Roussou E, Scarpato S, Sorensen I J, Valle-Onate R, Weber U, Wei J, Sieper J. The Assessment of SpondyloArthritis International Society classification criteria for peripheral spondyloarthritis and for spondyloarthritis in general. Ann Rheum Dis. 2011;70(1):25-31. doi: 10.1136/ard.2010.133645. PubMed PMID: 21109520.
51. Machado P M, Landewé R B, van der Heijde D M. Endorsement of definitions of disease activity states and improvement scores for the Ankylosing Spondylitis Disease Activity Score: results from OMERACT 10. J Rheumatol. 2011;38(7):1502-6. doi: 10.3899/jrheum. 110279. PubMed PMID: 21724723.
52. Park S H, Choe J Y, Kim S K, Lee H, Castrejón I, Pincus T. Routine Assessment of Patient Index Data (RAPID3) and Bath Ankylosing Spondylitis Disease Activity Index (BASDAI) Scores Yield Similar Information in 85 Korean Patients With Ankylosing Spondylitis Seen in Usual Clinical Care. J Clin Rheumatol. 2015;21(6):300-4. doi: 10.1097/RHU.0000000000000277. PubMed PMID: 26308349; PMCID: PMC4629489.
53. Turnbaugh P J, Ridaura V K, Faith J J, Rey F E, Knight R, Gordon J I. The effect of diet on the human gut microbiome: a metagenomic analysis in humanized gnotobiotic mice. Sci Transl Med. 2009;1(6):6ra14. doi: 10.1126/scitranslmed.3000322. PubMed PMID: 20368178; PMCID: PMC2894525.
54. Vermeire S, Schreiber S, Sandborn W J, Dubois C, Rutgeerts P. Correlation between the Crohn's disease activity and Harvey-Bradshaw indices in assessing Crohn's disease severity. Clin Gastroenterol Hepatol. 2010;8(4):357-63. Epub 2010 Jan. 21. doi: 10.1016/j.cgh.2010.01.001. PubMed PMID: 20096379.
55. Bolger A M, Lohse M, Usadel B. Trimmomatic: a flexible trimmer for Illumina sequence data. Bioinformatics. 2014;30(15):2114-20. Epub 2014 Apr. 1. doi: 10.1093/bioinformatics/btu 170. PubMed PMID: 24695404; PMCID: PMC4103590.
56. Truong D T, Franzosa E A, Tickle T L, Scholz M, Weingart G, Pasolli E, Tett A, Huttenhower C, Segata N. MetaPhlAn2 for enhanced metagenomic taxonomic profiling. Nat Methods. 2015;12(10):902-3. doi: 10.1038/nmeth.3589. PubMed PMID: 26418763.
57. Langmead B, Salzberg S L. Fast gapped-read alignment with Bowtie 2. Nat Methods. 2012;9(4):357-9. Epub 2012 Mar. 4. doi: 10.1038/nmeth. 1923. PubMed PMID: 22388286; PMCID: PMC3322381.
58. Abubucker S, Segata N, Goll J, Schubert A M, Izard J, Cantarel B L, Rodriguez-Mueller B, Zucker J, Thiagarajan M, Henrissat B, White O, Kelley S T, Methé B, Schloss P D, Gevers D, Mitreva M, Huttenhower C. Metabolic reconstruction for metagenomic data and its application to the human microbiome. PLoS Comput Biol. 2012;8(6):e1002358. Epub 2012 Jun. 13. doi: 10.1371/journal.pcbi.1002358. PubMed PMID: 22719234; PMCID: PMC3374609.
59. Buchfink B, Xie C, Huson D H. Fast and sensitive protein alignment using DIAMOND. Nat Methods. 2015;12(1):59-60. Epub 2014 Nov. 17. doi: 10.1038/nmeth.3176. PubMed PMID: 25402007.
60. Suzek B E, Wang Y, Huang H, McGarvey P B, Wu C H, Consortium U. UniRef clusters: a comprehensive and scalable alternative for improving sequence similarity searches. Bioinformatics. 2015;31(6):926-32. Epub 2014 Nov. 13. doi: 10.1093/bioinformatics/btu739. PubMed PMID: 25398609; PMCID: PMC4375400.
61. Caspi R, Billington R, Fulcher C A, Keseler I M, Kothari A, Krummenacker M, Latendresse M, Midford PE, Ong Q, Ong W K, Paley S, Subhraveti P, Karp P D. The MetaCyc database of metabolic pathways and enzymes. Nucleic Acids Res. 2018;46(D1):D633-D9. doi: 10.1093/nar/gkx935. PubMed PMID: 29059334; PMCID: PMC5753197.
62. Lu Y, Yao D, Chen C. 2-Hydrazinoquinoline as a Derivatization Agent for LC-MS-Based Metabolomic Investigation of Diabetic Ketoacidosis. Metabolites. 2013;3(4):993-1010. doi: 10.3390/metabo3040993. PubMed PMID: 24958262; PMCID: PMC3937830.
63. Callahan B J, McMurdie P J, Rosen M J, Han A W, Johnson A J, Holmes S P. DADA2: High-resolution sample inference from Illumina amplicon data. Nat Methods. 2016;13(7):581-3. Epub 2016 May 24. doi: 10.1038/nmeth.3869. PubMed PMID: 27214047; PMCID: PMC4927377.
64. Rognes T, Flouri T, Nichols B, Quince C, Mahe F. VSEARCH: a versatile open source tool for metagenomics. PeerJ. 2016;4: e2584. Epub 2016 Oct. 27. doi: 10.7717/peerj.2584. PubMed PMID: 27781170; PMCID: PMC5075697.
65. Quast C, Pruesse E, Yilmaz P, Gerken J, Schweer T, Yarza P, Peplies J, Glöckner F O. The SILVA ribosomal RNA gene database project: improved data processing and web-based tools. Nucleic Acids Res. 2013;41(Database issue):D590-6. Epub 20121128. doi: 10.1093/nar/gks1219. PubMed PMID: 23193283; PMCID: PMC3531112.
66. McMurdie P J, Holmes S. phyloseq: an R package for reproducible interactive analysis and graphics of microbiome census data. PLoS One. 2013;8(4):e61217. doi: 10.1371/journal.pone.0061217. PubMed PMID: 23630581; PMCID: PMC3632530.
67. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013;29(1):15-21. Epub 20121025. doi: 10.1093/bioinformatics/bts635. PubMed PMID: 23104886; PMCID: PMC3530905.
68. Hartley S W, Mullikin J C. QoRTs: a comprehensive toolset for quality control and data processing of RNA-Seq experiments. BMC Bioinformatics. 2015;16(1):224. Epub 20150719. doi: 10.1186/s12859-015-0670-5. PubMed PMID: 26187896; PMCID: PMC4506620.
69. Liao Y, Smyth G K, Shi W. featureCounts: an efficient general purpose program for assigning sequence reads to genomic features. Bioinformatics. 2014;30(7):923-30. Epub 20131113. doi: 10.1093/bioinformatics/btt656. PubMed PMID: 24227677.
70. Karp P D, Billington R, Caspi R, Fulcher C A, Latendresse M, Kothari A, Keseler I M, Krummenacker M, Midford P E, Ong Q, Ong W K, Paley S M, Subhraveti P. The BioCyc collection of microbial genomes and metabolic pathways. Brief Bioinform. 2019;20(4):1085-93. doi: 10.1093/bib/bbx085. PubMed PMID: 29447345; PMCID: PMC6781571.
71. Walters W, Hyde E R, Berg-Lyons D, Ackermann G, Humphrey G, Parada A, Gilbert J A, Jansson J K, Caporaso J G, Fuhrman J A, Apprill A, Knight R. Improved Bacterial 16S IRNA Gene (V4 and V4-5) and Fungal Internal Transcribed Spacer Marker Gene Primers for Microbial Community Surveys. mSystems. 2016;1(1). Epub 20151222. doi: 10.1128/mSystems.00009-15. PubMed PMID: 27822518; PMCID: PMC5069754.
72. Love M I, Huber W, Anders S. Moderated estimation of fold change and dispersion for RNA-seq data with DESeq2. Genome Biol. 2014;15(12):550. doi: 10.1186/s13059-014-0550-8. PubMed PMID: 25516281; PMCID: PMC4302049.

What is claimed is:

1. A method for treating an individual who has inflammatory bowel diseases (IBD) and spondyloarthritis, the method comprising:
i) selecting the individual based on a determination from a biological sample obtained from the individual that the gastrointestinal system of the individual comprises a microbiome lacking bacteria that provide functional folate trap;
ii) administering to the individual selected as in i) sulfasalazine with bacteria comprising a functional folate trap to thereby treat the IBD.

2. The method of claim 1, wherein the bacteria comprising the functional folate trap that are administered to the individual include Faecalibacterium prausnitzii.

3. The method of claim 2, wherein the bacteria comprising the functional folate trap that are administered to the individual further include Ruminococcus Callidus.

4. The method of claim 3, wherein the bacteria comprising the functional folate trap that are administered to the individual further comprise at least one of Alistipes onderdonkii, Bacteroides nordii, Collinsella aerofaciens, or Roseburia imulinivorans.

5. The method claim 4, wherein the method comprises analyzing a biological sample obtained from the individual to determine that the gastrointestinal system of the individual comprises a microbiome lacking bacteria that provide the functional folate trap prior to administering the sulfasalazine and prior to administering the bacteria comprising the functional folate trap.

6. The method of claim 5, wherein the biological sample comprises a fecal sample.

7. The method of claim 6, wherein determining the gastrointestinal system of the individual comprises a microbiome lacking the bacteria that provide the functional folate trap comprises measuring relative abundance of F. prausnitzii in the microbiome with shotgun metagenomic sequencing of the microbiome or 16S ribosomal RNA based polymerase chain reaction. 8 The method of claim 6, wherein determining the gastrointestinal system of the individual comprises a microbiome lacking the bacteria that provide the functional folate trap comprises measuring baseline thymine fecal levels or baseline fecal deoxyuridine levels, or a combination thereof.


SEQUENCE LISTING (6 sequences)

SEQ ID NO: 1 - DNA, 18 bp, synthetic construct
  source 1..18: mol_type = other DNA
tgycagcmgccgcggtaa

SEQ ID NO: 2 - DNA, 20 bp, synthetic construct
  source 1..20: mol_type = other DNA
ccgycaattymtttragttt

SEQ ID NO: 3 - DNA, 22 bp, synthetic construct
  source 1..22: mol_type = other DNA
gtggatgcctttgtggatattg

SEQ ID NO: 4 - DNA, 22 bp, synthetic construct
  source 1..22: mol_type = other DNA
ccgggttatcgttcaggtaatc

SEQ ID NO: 5 - DNA, 22 bp, synthetic construct
  source 1..22: mol_type = other DNA
ctcagcagatgaggcagtaaag

SEQ ID NO: 6 - DNA, 22 bp, synthetic construct
  source 1..22: mol_type = other DNA
ggaagctctgagtaacggattg